(19) United States
(12) Patent Application Publication
(10) Pub. No.: US 2008/0085874 A1
(43) Pub. Date: Apr. 10, 2008

(54) SMALL MOLECULE POTENTIATOR OF HORMONAL THERAPY FOR BREAST CANCER

(75) Inventors: Peter Kushner, San Francisco, CA (US); Ira D. Goldfine, Tiburon, CA (US); Leslie Hodges-Gallagher, San Francisco, CA (US); Cathleen D. Valentine, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California

(21) Appl. No.: 11/845,554

(22) Filed: Aug. 27, 2007

Related U.S. Application Data
(60) Provisional application No. 60/840,741, filed on Aug. 28, 2006.
(60) Provisional application No. 60/911,431, filed on Apr. 12, 2007.

(51) Int. Cl. A61K 31/135; A61K 31/41; A61K 31/445; A61K 31/55; A61K 31/56; A61P 43/00

(52) U.S. Cl. 514/177; 514/182; 514/217; 514/324; 514/383; 514/648

(57) ABSTRACT

The present application demonstrates that HDAC inhibitors can be used in combination with hormonal therapy to treat and prevent estrogen receptor positive breast cancer. HDAC inhibitors can also be combined with IGF-1R inhibitors, mTOR inhibitors, and EGFR inhibitors to treat breast cancer, optionally in combination with hormonal therapy if indicated. Combinations of the compounds, with or without HDAC inhibitors, and with or without hormonal therapy, can also be used. The invention therefore provides methods of treatment and pharmaceutical compositions.

### CROSS-REFERENCES TO RELATED APPLICATIONS

The present application is related to U.S. Ser. No. 60/840,741, filed Aug. 28, 2006, and to U.S. Ser. No. 60/911,431, filed Apr. 12, 2007, each incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under National Cancer Institute NIH R01 CA 80210. The US Government has certain rights in this invention.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

Although many breast cancer therapies exist, there is a need to develop therapeutics that are safe and effective, and which circumvent resistance against hormonal and other therapies in breast tumors, that do not cause increases in other types of cancer, and which extend the disease-free survival of patients. For example, while the majority of patients with ERα-positive breast tumors initially respond favorably to antiestrogen therapy with tamoxifen or fulvestrant, or to estrogen ablation therapy with aromatase inhibitors, most tumors eventually acquire resistance to these hormonal therapies despite maintaining ERα expression (Clarke, R., et al., J Steroid Biochem Mol. Biol., 2001. 76(1-5): p. 71-84; Osborne, C. K., N Engl J. Med., 1998. 339(22): p. 1609-18; Ring, A. and M. Dowsett, Endoer Relat Cancer., 2004. 11(4): p. 643-58). In addition, tamoxifen therapy has the undesirable side effect of stimulating proliferation of uterine endometrial cells, putting women at a higher risk for developing uterine adenocarcinoma (Fisher, B., et al., B-14. J Natl Cancer Inst, 1994. 86(7): p. 527-37). One therapeutic strategy is to combine hormonal therapies that target ERα-driven proliferation with agents that target separate biochemical pathways to determine if the combination would provide enhanced and more long-lived efficacy. Although several randomized trials have combined antiestrogens simultaneously with traditional adjuvant chemotherapy, these trials have produced disappointing results, perhaps because hormonal therapy can antagonize the effectiveness of chemotherapy, leading to the result that these combinations are no more or even less effective when combined than when the individual therapeutic compounds are administered separately (Gelber, R. D., et al., Lancet, 1996. 347(9008): p. 1066-71; Rivkin, S. E., et al., J Clin Oncol, 1994. 12(10): p. 2078-85). Such antagonist action of hormonal therapy is seen in cell culture, and may result both from antiestrogen effects on cell cycling and independent actions (Woods, K. E., J. K. Randolph, and D. A. Gewirtz, Biochem Pharmacol, 1994. 47(8): p. 1449-52; De Soto, J. A., et al., Anticancer Res, 2002. 22(2A): p. 1007-9; Osborne, C. K., L, Kitten, and C I. Arteaga, J. Clin Oncol, 1989. 7(6): p. 710-7).

A more effective strategy, therefore, may be to combine hormonal therapy with a second therapy whose target is different from standard chemotherapy, so that the combined effects are more effective than any of the individual compounds administered separately. Studies combining various therapies have shown some promise, though generally there is confusion in the literature (Johnston, S. R., Clin Cancer Res, 2006. 12(3 Pt 2): p. 1061s-1068s). For example, a recent trial of the combination of the aromatase inhibitor letrozole with temsirolimus, an inhibitor of mTOR, the downstream effector of IGF signaling, was terminated for lack of benefit (Termination of phase 3 clinical program with oral temsirolimus in women with metastatic breast cancer. Press release, Wyeth Pharmaceuticals, 2006).

Histone deacetylase (HDAC) inhibitors are a structurally diverse group of pharmacological agents that inhibit proliferation, induce differentiation and/or apoptosis in a wide range of cancer cells and hold much promise as anti-neoplastic agents (Villar-Garea, A. and M. Esteller, Int J Cancer, 2004. 112(2): p. 171-8; Marks, P., et al., Nat Rev Cancer, 2001. 1(3): p. 194-202). Hyperacetylation is associated with a transcriptionally permissive environment and HDAC inhibitors, although they affect only a small number of target genes, activate genes involved in cell cycle arrest, apoptosis and differentiation (Glaser, K. B., et al., Mol Cancer Ther, 2003. 2(2): p. 151-63; Richon, V. M., et al., Proc Natl Acad Sci USA, 2000. 97(18): p. 10014-9; Munster, P. N., et al., Cancer Res., 2001. 61(23): p. 8492-7). Furthermore, HDAC inhibitors increase the efficiency of several anticancer drugs that target the DNA (Kim, M. S., et al., Cancer Res, 2003. 63(21): p. 7291-300; Castro-Galache, M. D., et al., Int J Cancer, 2003. 104(5): p. 579-86). A variety of small molecule HDAC inhibitors are currently in preclinical development.

The clinical effects of combining HDAC inhibitors with other therapies, such as antiestrogen therapy in ERα-positive breast tumors are uncertain; an increase in antiestrogen driven ERα activity at the transcriptional level could potentially result in partial agonist activity at the proliferative level (see, for e.g., Munster, P. N., et al., Cancer Res., 2001. 61(23): p. 8492-7; Vigushin, D. M., et al., Clin Cancer Res., 2001. 7(4): p. 971-6; Margueron, R., et al., J. Endocrinol., 2003. 179(1): p. 41-53 Webb, P., P. Nguyen, and P. J. Kushner, J Biol Chem, 2003. 278(9): p. 6912-20; Jang, E. R., et al., Oncogene 2004. 23(9): p. 1724-36; Margueron, R., et al, J. Mol. Endocrinol., 2004, 32(2): p. 583-94). In addition, there are reports suggesting against the combination of HDAC inhibitors with hormonal therapy (e.g., Jansen, M. et al., 2004. Proc Natl Acad Sci USA 101:7199-20).

There is a compelling need, therefore, to develop new therapeutic strategies for the treatment and prevention of cancer, and in particular, for the treatment and prevention of breast cancer.

### SUMMARY OF THE INVENTION

The present inventors have therefore investigated whether valproic acid (VPA), carbamazepine, and other HDAC inhibitors combine effectively with hormonal therapy, including antiestrogens and aromatase inhibitors, on human ERα-positive breast cancer cells. The inventors have discovered that surprisingly, certain combinations of HDAC inhibitors combine effectively with hormonal therapy, and that the combinations do not reduce the activity of any single component, and that they combinations are more effective than either of the components alone. The inventors have also investigated the effects of various combinations of HDAC inhibitors in combination with mTOR inhibitors, EGFR inhibitors, and IGF-1R inhibitors, with and without hormonal therapy. The findings indicate that combination with VPA combines effectively with the inhibitory actions of antiestrogens and aromatase inhibitors, and unlike compounds traditionally used in combination in the treatment and prevention of breast cancer, these combinations do not reduce the effectiveness compared to that of the individual components. As an added benefit, HDAC inhibitors, such as VPA, counters the pro-proliferative action of tamoxifen on uterine cells. The findings also indicate that HDAC inhibitors work effectively in combination with mTOR inhibitors, EGFR inhibitors, or IGF-1R inhibitors to treat breast cancer and further in combination with hormone therapy to treat estrogen receptor positive breast cancer. Furthermore, VPA is effective in combining with tamoxifen in cells rendered tamoxifen-resistant by overexpression of HER2/neu.

Thus, the present inventors have found that certain combinations of HDAC inhibitors, hormonal therapy agents, and other compounds, including, but not limited to IGF-1R inhibitors, EGFR inhibitors, and mTOR inhibitors combine effectively with each other and are superior to other combinations of compounds for the treatment and prevention of breast cancer, and for preventing the progression of breast cancer. The present invention provides methods of treating and preventing estrogen receptor positive breast cancer, as well as pharmaceutical compositions comprising the compounds used in the combination therapies.

The present invention encompasses methods of treatment for (including management of, amelioration of symptoms of, and preventing the progression of) breast cancer, using certain combination therapies, as well as the pharmaceutical compositions comprising these combination therapies. The invention is based, in part, on the recognition that certain combinations of compounds combine effectively with each other, and are superior to other combinations of compounds, as well as improving the tolerance of, and/or reducing the side effects caused by at least one of the compounds in the combination. Subjects are mammalian, and preferably are human, and more preferably are human females.

In one aspect, the present invention provides methods of treating and methods of preventing estrogen receptor positive breast cancer, comprising administering to a subject a therapeutically effective amount of an HDAC inhibitor in combination with a course of hormonal therapy. In certain embodiments, the HDAC inhibitor is not VPA, carbamazepine, or SAHA.

The present invention also encompasses methods and compositions for preventing breast cancer, particularly in subjects who are at risk for breast cancer that is greater than the average risk for breast cancer. Risk factors considered in preventing breast cancer in subjects include family history of breast cancer (relatives with breast cancer), genetic markers for breast cancer such as BRCA1 and BRCA2, age at menarche, age at first live birth, the number of breast biopsies, presence of atypical hyperplasia on breast biopsy, population rates of breast cancer and death from other causes. The present invention also provides methods and compositions for preventing the progression of breast cancer to a later stage for those who already have breast cancer or precancerous indicators, as well as preventing the recurrence of breast cancer for those in remission from breast cancer.

In some embodiments, the invention contemplates methods of preventing the progression of DCIS to breast cancer, and methods of preventing the progression of atypical hyperplasia to breast cancer. In some preferred embodiments, the invention encompasses treating or preventing estrogen receptor positive breast cancer.

In yet another embodiment, the present invention encompasses treating DCIS and, in another embodiment, the present invention encompasses treating atypical hyperplasia.

The present invention also encompasses methods of treating and methods of preventing breast cancer comprising administering to a subject suffering therefrom a therapeutically effective amount of an HDAC inhibitor in combination with a therapeutically effective amount of one or more of an IGF-1 receptor inhibitor, an EGFR inhibitor, or an mTOR inhibitor. In certain embodiments, the HDAC inhibitor is not VPA when in combination with an EGFR inhibitor.

In a further aspect of the invention, methods are provided for treating and for preventing breast cancer, comprising administering to a subject suffering therefrom a therapeutically effective amount of an HDAC inhibitor in combination with a course of hormonal therapy as well as an additional active ingredient that is effective to treat breast cancer in the combination. In some embodiments, the method comprises administering a combination comprising an HDAC inhibitor, a course of hormonal therapy, and one or more of an IGF-1 receptor inhibitor, an EGFR inhibitor, or an mTOR inhibitor.

In yet another aspect of the invention, methods are provided for treating and for preventing breast cancer, comprising administering to a subject suffering therefrom a therapeutically effective amount of a course of hormonal therapy in combination with one or more of an IGF-1 receptor inhibitor, an EGFR inhibitor, or an mTOR inhibitor.

In yet another aspect of the invention, methods are provided for treating and for preventing breast cancer, comprising administering to a subject suffering therefrom a therapeutically effective amount a combination of two or more of an IGF-1 receptor inhibitor, an EGFR inhibitor, or an mTOR inhibitor.

The present invention also provides pharmaceutical compositions comprising or consisting essentially of the combinations of compounds described herein, as well as kits comprising the combinations.

The HDAC inhibitors encompassed by the present invention can be any known to those of skill in the art, particularly those shown in FIGS. 8A-8F and those described in Minucci et al, Nature 6:38-51 (2006). According to the invention, in some embodiments, the HDAC inhibitor is carbamazepine, and in other embodiments the HDAC inhibitor is valproic acid, either the free acid or the sodium or magnesium salt. In yet other embodiments, the HDAC inhibitor may be TSA, SAHA, or any other HDAC inhibitor known by the skilled practitioner to be effective. In one embodiment, the patient is treated with a combination of tamoxifen and VPA. In another embodiment, the patient is treated with tamoxifen, VPA, and EGCG. In another embodiment, the patient is treated with tamoxifen, VPA, and rapamycin.

In one embodiment, the daily dose of valproic acid is from about 15/mg/kg to about 60 mg/kg.

In one embodiment, the daily dose of valproic acid is sufficient to achieve about 300 to about 867 micromolar in patient serum. In another embodiment, the daily dose of valproic acid is sufficient to achieve about 300 to about 1000 micromolar in patient serum, and in another embodiment, the daily dose of valproic acid is sufficient to achieve about 500 to about 1000 micromolar in patient serum.

In some embodiments, the dose of carbamazepine is from about 800 mg/day to about 1600 mg/day. In other embodiments, the dose of carbamazepine is from about 800 mg/day to about 1200 mg/day. In yet other embodiments, the dose of carbamazepine is from about 200 mg/day to about 600 mg/day.

In some embodiments, the dose of SAHA is from about 200 mg/day to about 600 mg/day. In another embodiment, the dose of SAHA is about 400 mg/day.

In some embodiments, the hormonal therapy is anti-estrogen therapy, which can be, but is not limited to, tamoxifen, raloxifene, fulvestrant, or toremifene. The hormonal therapy can also be estrogen ablation therapy, including an aromatase inhibitor. According to the invention, the aromatase inhibitor can be, but is not limited to, exemestane, letrozole, or anastrozole.

In some embodiments, the dose of tamoxifen is from about 10 mg/day to about 50 mg/day. In another embodiment, the dose of tamoxifen is about 20 mg/day. In one embodiment in which a patient has metastatic breast cancer, the dose of tamoxifen is from about 20 mg/day to about 40 mg/day.

In one embodiment, the dose of letrozole is from about 1 mg/day to about 5 mg/day. In another embodiment, the dose of letrozole is about 2.5 mg/day.

In one embodiment, the dose of exemestane is from about 10 mg/day to about 40 mg/day. In another embodiment, the dose of exemestane is about 25 mg/day.

In yet another embodiment, the dose of anastrozole is from about 0.5 mg/day to about 3 mg/day. In another embodiment, the dose of anastrozole is about 1 mg/day.

In some embodiments, compounds are administered in combination, with ratios of those compounds which preserve the recommended daily doses of the compounds. In some embodiments, compounds are administered in combination, with ratios of those compounds which preserve the ranges of doses as described herein.

In one embodiment, the ratio of tamoxifen to VPA is 1 part tamoxifen to 45-180 parts VPA, for a 60 kg patient.

In another embodiment, where the patient has metastatic breast cancer, the ratio of tamoxifen to VPA is 1 part tamoxifen to 22.5-180 parts VPA, for a 60 kg patient.

When the combination comprises administering tamoxifen or raloxifen along with an HDAC inhibitor, there is a reduction in or no attendant increase in the risk of uterine cancer. Treatment with tamoxifen or raloxifene is compromised by an increased risk in uterine cancer. A distinct advantage of the present invention is that administration of an HDAC inhibitor in combination with tamoxifen or raloxifene reduces or eliminates the risk of uterine cancer. In some embodiments, this risk is reduced by 10%, 20%, 30%, 40%, 50%, 60%, 70%, 80%, 90%, 95%, or it is virtually or wholly eliminated.

The present invention further contemplates that the combination therapies as described herein can also reduce or eliminate other side effects of treatment, at least in part because lower doses of compounds can be used in treatment or prevention protocols.

In certain of the embodiments, the IGF-1R inhibitor can be picropodophyllin (see, e.g., Girnita, A. et al., Cancer Res., 2004. 64(1): 236-242) or the green tea polyphenol, EGCG (see, e.g., Shimizu, M. et al, Biochem. Biophys. Res. Commun., 2005. 334(3): 947-953; Li, M. et al., Cancer Epidemiol. Biomarkers Prev., 2007. 16(3): 598-605. The EGFR inhibitor may be gefitinib, and the mTOR inhibitor may be rapamycin or rapamycin derivatives (see, e.g., Johnston, S. R., Clin. Cancer Res., 2006. 12(3 Pt. 2): 1061-1069s). The skilled practitioner will be able to use a variety of IGF-1R, EGFR, and mTOR inhibitors in the invention, to provide therapeutically effective combinations with various HDAC inhibitors.

In one embodiment, the dose of EGCG is from about 300 mg/day to about 800 mg/day.

In one embodiment, the dose of rapamycin is from about 0.125 mg/day to about 1 mg/day.

In one embodiment, the dose of gefitinib is from about 200 mg/day to about 300 mg/day. In another embodiment, the dose of gefitinib is about 250 mg/day.

In one embodiment, the dose of erlotinib is from about 100 mg/day to about 150 mg/day.

The additional “active ingredient” that can be used in combination with an HDAC inhibitor and hormonal therapy can be chosen from among a variety of compounds. In different embodiments, the additional active ingredient can be an IGF-1R inhibitor, an EGFR inhibitor, an mTOR inhibitor, or other chemotherapeutic agent, biologic, radiation therapy, or other agents and procedures useful in the treatment of cancer.

Various drug administration protocols are contemplated by the invention. In some embodiments, the HDAC inhibitor is administered on a daily basis, while the hormonal therapy, or IGF-1R inhibitor, EGFR inhibitor, or mTOR inhibitor is administered every other day. In other embodiments, the hormonal therapy or IGF-1R inhibitor, EGFR inhibitor, or mTOR inhibitor is administered on a daily basis and the HDAC inhibitor is administered every other day. The invention also contemplates administering the HDAC inhibitor and the hormonal therapy or IGF-1R inhibitor, EGFR inhibitor, or mTOR inhibitor concurrently.

It is within the scope of the invention to treat breast cancer that is tamoxifen resistant, as well as to treat breast cancer that overexpresses Her2/Neu. The invention also contemplates treating subjects with breast cancer for whom previous therapy has failed, or for whom the cancer is recurring. In some embodiments of the invention, the invention is to treat subjects with breast cancer who are post-menopausal, and in some embodiments, the invention contemplates treating subjects who are genetically predisposed to breast cancer or otherwise at increased risk. The invention also encompasses methods of treating subjects to prevent progression of breast cancer, and in some embodiments, the invention encompasses treating or preventing breast cancer in patients with pre-cancerous growths or benign tumors. It is within the scope of the invention to treat subjects that are in remission from breast cancer, and to treat subjects with breast cancer that have previously undergone treatment.

### BRIEF DESCRIPTION OF THE DRAWINGS

FIGS. 1A-1C: VPA inhibits cell proliferation and enhances the antiproliferative effect of tamoxifen in ERα positive breast cancer cells. MCF-7 (A), T47D (B) and ZR-75-1 (C) cells were grown for 6-7 days and counted electronically. Cells were treated with 750 μM VPA, 10 nM OH-Tam and 100 μM E2, as indicated. Bars represent the average of three independent experiments presented as a percentage of E2 alone and error bars represent S.E.M. from the three experiments. Statistical significance was determined by ANOVA at p≦0.05 with a denoting statistical difference from E2 alone; b denoting statistical difference from E+VPA+OH-Tam; and c denoting statistical difference from vehicle alone; and d denoting statistical difference from VPA+OH-Tam.

FIGS. 2A-2C: VPA enhances the efficacy of tamoxifen and other antiestrogens. VPA and OH-Tam have an additive effect in inhibiting MCF-7 cell proliferation (A). Cells were treated with 100 pM E2 with and without 750 μM VPA in the presence of OH-Tam, ranging from 0 to 500 nM. On day 6 of the assay, proliferation was measured using a fluorescent DNA-binding assay and values represent the percentage of fluorescence of E2 alone (control) with bars representing the S.E.M. from triplicate wells. VPA enhances the inhibition of cell growth of two other antiestrogens, raloxifene and fulvestrant (B). MCF-7 cells were treated with OH-Tam, raloxifene (Ral) or fulvestrant (Fulv) in the presence of 100 pM E2 either plus or minus 750 μM VPA for 7 days and electronically counted. Bars represent the average proliferative response relative to E2 alone (control) from three independent experiments with error bars representing S.E.M. VPA enhances the efficacy of letrozole (C). MCF-7aro cells were treated with either 100 nM E2 or 1 nM testosterone (T) with the indicated concentrations of letrozole (Let) for 5 days with or without 750 μM VPA and assayed as described above.

FIGS. 3A-3C: HDAC inhibitors enhance tamoxifen antiproliferative action. MCF-7 cells were treated in the presence of 100 pM E2 with a range of doses of VPA (A), TSA (B) or SAHA (C), alone and in the presence of 10 nM OH-Tam. Proliferation was measured on day 7 using a fluorescent DNA-binding assay and values represent the percentage of fluorescence of 100 pM E2 alone (control) and bars represent the S.E.M. from triplicate wells.

FIGS. 4A-4F: VPA enhances the antiproliferative effect of tamoxifen primarily by increasing apoptosis. MCF-7 cells were treated with 100 pM E2 alone (A) and with 7501 μM VPA (B), or 10 nM OH-Tam (C) or a combination of both VPA and OH-Tam (D) for a 6-day period and a representative field analyzed by phase microscopy at 10× magnification. The arrow points to a group of cells with apoptotic-like morphology. VPA alone or in combination with OH-Tam does not alter E2-induced cell cycle distribution (E). MCF-7 cells were treated for 48 hours as described above and DNA content measured by flow cytometry. VPA increases apoptosis and enhances tamoxifen-induced apoptosis (F). MCF-7 cells were treated for 72 hours in the absence of E2 and apoptotic index measured by AnnexinV-fluorescein staining. A minimum of 2500 nuclei were analyzed for each treatment from a total of two independent experiments.

FIGS. 5A-5B: Effect of VPA on tamoxifen-induced gene expression. VPA activates transcription and enhances tamoxifen activity of an ERE reporter gene (A). MCF-7 cells were transiently transfected with ERE-Luc and treated with and without 10 nM OH-Tam along with the indicated concentrations of ligands for 24 hours and assayed for lucifererase activity. Bars represent fold-induction relative to vehicle from a representative experiment and error bars represent the S.E.M. from triplicate wells. VPA and tamoxifen cooperate in upregulating the pro-apoptotic protein Bik, (B). MCF-7 cells ere treated for 72 hours with vehicle or 100 pM E2, 750 μM VPA, and/or 10 nM OH-Tam and protein lysates immunoblotted with ERα, CD 1, Bik, or Bcl-2 antibodies, with β-tubulin serving as a loading control.

FIG. 6: VPA enhances the efficacy of tamoxifen in MCF-7 cells overexpressing HER2/neu. MCF-7/neo and MCF-7/HER2 cells were treated with 10 pM E2 and either 750 μM VPA and/or 10 nM OH-Tam for 7 days and counted electronically. Bars represent the average cell number of three replicates from a representative experiment and error bars represent S.E.M.

FIG. 7: VPA antagonizes tamoxifen-induced proliferation in endometrial cells. Ishikawa endometrial adenocarcinoma cells were grown for 6-7 days and counted electronically. Cells were treated with 750 μM VPA, 10 nM OH-Tam and 100 pM or 1 nM E2, as indicated. Bars represent the average of three experiments and error bars represent S.E.M.

FIGS. 8A-8F: These figures present representative HDAC inhibitors.

FIG. 9: This figure shows that carbamazepine, a HDAC inhibitor, combines effectively with rapamycin, picropodophyllin, and tamoxifen to slow breast cancer cell growth.

FIG. 10: This figure shows that valproic acid, a HDAC inhibitor, picropodophyllin, an IGF-1R inhibitor, and rapamycin, an mTOR inhibitor, combine effectively with each other and with tamoxifen to inhibit breast cancer cell growth.

FIG. 11: This figure shows that valproic acid combines effectively with gefitinib, an EGFR inhibitor, and with rapamycin, but also shows that the latter two drugs fail to combine effectively with each other to inhibit breast cancer cell growth.

FIGS. 12A-12C: Effect of the combination of HDAC inhibitors (valproic acid in (A), TSA in (B), and carbamazepine in (C)) with EGCG and rapamycin, with or without tamoxifen, on the inhibition of breast cancer cells. Combinations of the four agents are more efficacious than treatment with any single agent alone.

### DETAILED DESCRIPTION OF THE INVENTION

The present inventors have found that certain combinations of HDAC inhibitors, hormonal therapy agents, and other compounds, including, but not limited to IGF-1R inhibitors, EGFR inhibitors, and mTOR inhibitors combine effectively with each other for the treatment and prevention of breast cancer, and for preventing the progression of breast cancer. The inventors have discovered that, surprisingly, certain combinations of HDAC inhibitors combine effectively with hormonal therapy, and that the combinations are more effective than either of the components alone. The present invention provides methods of treating and preventing estrogen receptor positive breast cancer, as well as pharmaceutical compositions comprising the compounds used in the combination therapies disclosed herein.

The present invention is based on the discovery that VPA, which is an HDAC inhibitor, enhances the anti-proliferative effect of tamoxifen in three estrogen receptor alpha (ERα)-positive breast cancer cells lines, MCF-7, T47-D and ZR-75-1. VPA also enhances the antiproliferative actions of two other antiestrogens, fulvestrant and raloxifene, as well as the antiproliferative effects of the aromatase inhibitor letrozole. Three other HDAC inhibitors, trichostatin A (TSA), carbamazepine, and suberoylanilide hydroxamic acid (SAHA), also enhance the efficacy of tamoxifen, indicating that cooperation, or effective combination, among HDAC inhibitors and antiestrogens may be a general phenomenon. VPA also increases tamoxifen sensitivity of a tamoxifen-resistant MCF-7 derivative cell line overexpressing HER2/neu. Remarkably, in addition to its ability to enhance the beneficial action of tamoxifen on breast cancer cells, VPA reverses the proliferative effect of tamoxifen in Ishikawa endometrial cells. Thus, the invention provides methods for treating both estrogen-sensitive tumors and tamoxifen-resistant breast tumors, while protecting the uterus from the negative proliferative effects observed with tamoxifen. The present invention also shows that the HDAC inhibitor, carbamazepine, combines effectively with rapamycin, an mTOR inhibitor, picropodophyllin, an IGF-1R inhibitor, and tamoxifen, to slow breast cancer cell growth. In addition, the present invention shows that VPA combines effectively with picropodophyllin and with rapamycin, as well as combining effectively with each other and with tamoxifen to inhibit breast cancer cell growth. Further, the present invention demonstrates that VPA combines effectively with gefitinib, an EGFR inhibitor, and with rapamycin, to slow breast cancer cell growth.

The invention also provides combinations of HDAC inhibitors, including but not limited to TSA, SAHA, valproic acid, and carbamazepine, as well as inhibitors of the mammalian target of rapamycin (mTOR) protein such as rapamycin or derivatives thereof, and inhibitor of the insulin-like growth factor receptor (IGF-1R) signaling pathway such as picropodophyllin, and inhibitors of EGFR. The combination of HDAC inhibitor, IGF-1R inhibitor, and mTOR inhibitor can also be used, as well as other combinations as described herein, including combinations with hormonal therapy. Each of these combinations can further be used in combination with hormone therapy, or other therapies to treat estrogen receptor positive breast cancer, as described herein.

In some embodiments, the individual compounds in the combination therapies combine effectively with each other, and in other embodiments, the individual compounds in the combination therapies synergize with each other.

### DEFINITIONS

As used herein, the term “cancer” refers to a disease involving cells that have the potential to metastasize to distal sites and exhibit phenotypic traits that differ from those of non-cancer cells. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells.

“Estrogen receptor positive breast cancer” refers to breast cancers that are in the positive or intermediate range for the estrogen receptor protein. For example, when estrogen receptor protein can be measured as femtomoles per milligram of cytosol protein. In this assay, values above 10 are positive, values from 3 to 10 are intermediate, and values less than 3 are negative. Other assays known in the art can be used to determined if the breast cancer is estrogen receptor positive, in particular assays based on antibodies to estrogen receptors alpha and beta and their use in biochemical or histological assays.

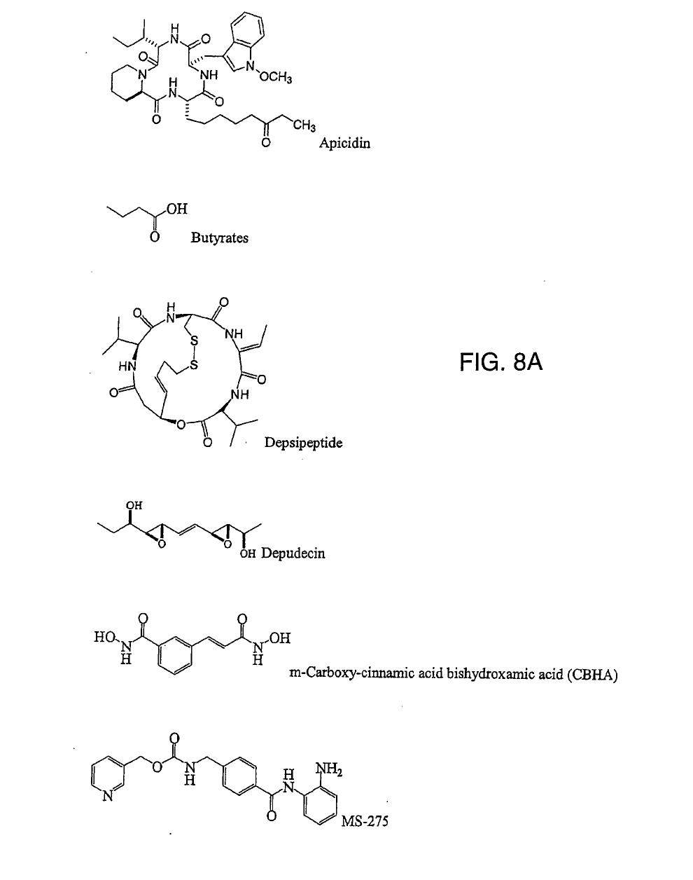

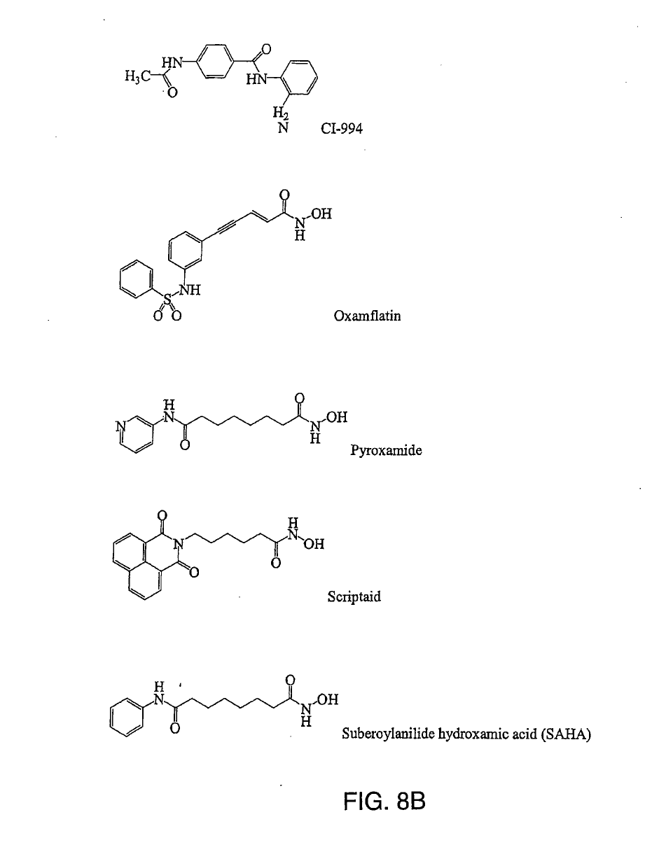

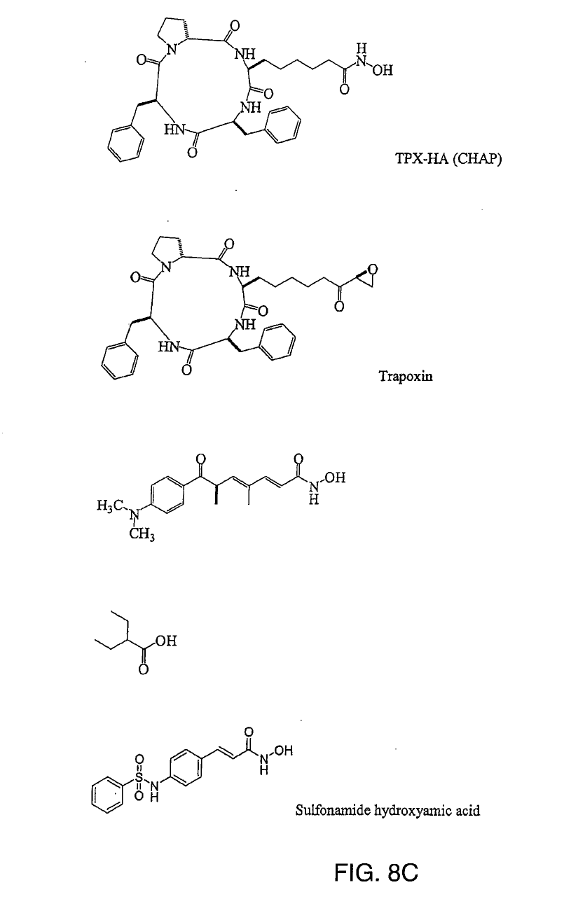

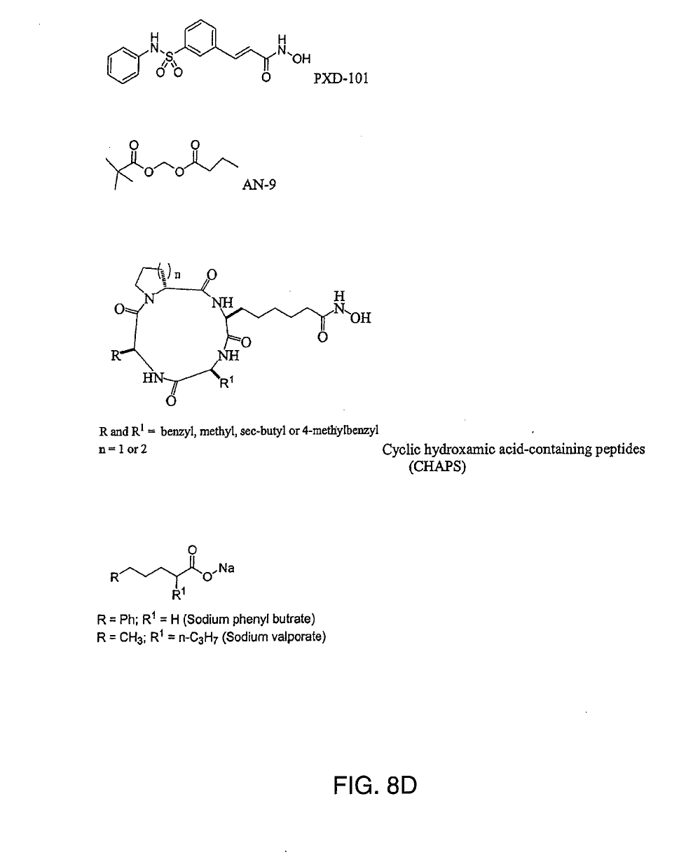

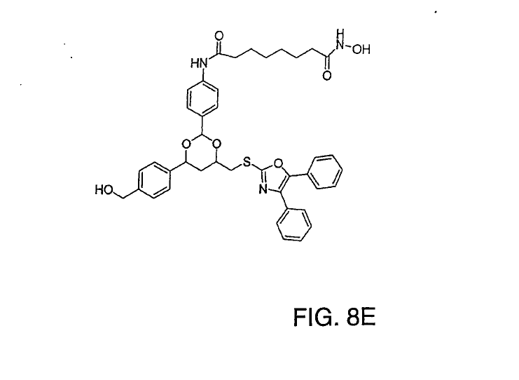

The terms “histone deacetylase inhibitor” and “inhibitor of histone deacetylase” mean a compound which is capable of interacting with a histone deacetylase and inhibiting its enzymatic activity. For examples, see the HDAC inhibitors in FIGS. 8A-8F. “Inhibiting histone deacetylase enzymatic activity” means reducing the ability of a histone deacetylase to remove an acetyl group from a histone. (see, e.g., FIG. 8 and Minucci et al., Nature 6:38-51 (2006). In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. Assays for determining inhibition are described in Phiel, C. J., et al., J Biol. Chem., 2001. 276(39): p. 36734-41 and Gottlicher, M., et al., Embo J., 2001. 20(24): p. 6969-78.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

As used herein, the term “active ingredient” includes having a therapeutic or prophylactic effect on breast cancer in the combinations. This does not include inactive ingredients such as pharmaceutical carriers, excipients, and the like.

“Mammalian target of rapamycin protein inhibitor” or “mTOR inhibitor” includes drugs such as rapamycin, temsirolimus, and everolimus that selectively inhibit the mammalian target of rapamycin (mTOR).

“IGF-1 receptor inhibitor” refers to drugs such as picrophodophyllin and podophyllotoxin that selectively inhibit the IGF-1 receptor.

“EGF receptor inhibitor” of “EGFR inhibitor” refers to drugs such as gefitinib and eroltinib that selectively inhibit the EGF receptor.

“Insufficient to fully prevent production of estrogen” refers to the inability of an aromatase inhibitor to fully prevent a tumor cell from converting an estrogen precursor into a functional estrogen that can stimulate tumor proliferation.

“Less than estrogen receptor-saturating amounts” refers to amounts of fulvestrant less than 100 fold molar excess to the amounts of estradiol or less than 10 nanomolar in patient circulation.

“Hormonal therapy” refers to drugs or treatments that block the effect of, or reduce the levels of hormones, and in particular which block the effect of estrogen or lower estrogen levels, including anti-estrogen therapy and estrogen ablation therapy.

As used herein, the terms “prevent,” “preventing” and “prevention” refer to the prevention of the recurrence, worsening, or spread of a disease in a subject resulting from the administration of a prophylactic or therapeutic agent.

The terms “overexpress,” “overexpression” or “overexpressed” interchangeably refer to a protein or nucleic acid (RNA) that is translated or transcribed at a detectably greater level, usually in a cancer cell, in comparison to a normal cell. The term includes overexpression due to transcription, post transcriptional processing, translation, post-translational processing, cellular localization (e.g., organelle, cytoplasm, nucleus, cell surface), and RNA and protein stability, as compared to a normal cell. Overexpression can be detected using conventional techniques for detecting mRNA (i.e., RT-PCR, PCR, hybridization, microarray) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

As used herein, the term “in combination” refers to the use of more than one prophylactic and/or therapeutic agents. The use of the term “in combination” does not restrict the order in which prophylactic and/or therapeutic agents are administered to a subject with cancer, especially breast cancer. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, hours, 96 hours, 1 week, 2 weeks, 3 weeks, 4 weeks, 5 weeks, 6 weeks, 8 weeks, or 12 weeks after) the administration of a second prophylactic or therapeutic agent to a subject which had, has, or is susceptible to cancer, especially breast cancer. The prophylactic or therapeutic agents are administered to a subject in a sequence and within a time interval such that the agent of the invention can act together with the other agent to provide an increased benefit than if they were administered otherwise. Any additional prophylactic or therapeutic agent can be administered in any order with the other additional prophylactic or therapeutic agents.

As used herein, the term “combine effectively” refers to a combination of therapies (e.g., a combination of prophylactic or therapeutic agents) which is more effective than any single agent administered alone. Combining effectively may also refer to combinations of therapies that are not less effective than any single agent or even less effective than any single agent, but which also eliminate or reduce the adverse effects of one or more of the agents, such as eliminating or reducing the risk of uterine cancer associated with one or more of the agents.

As used herein, the term “synergistic” refers to a combination of therapies (e.g., a combination of prophylactic or therapeutic agents) which is more effective than the additive effects of any two or more single agents. A synergistic effect of a combination of therapies permits the use of lower dosages of one or more of the therapies and/or less frequent administration of said therapies (e.g., agents) to a subject with a disease or disorder, in particular, cancer, or a condition or symptom associated therewith. The ability to utilize lower dosages of therapies and/or to administer said therapies less frequently reduces the toxicity associated with the administration of said therapies to a subject without reducing the efficacy of said therapies in the prevention, management, or treatment of a disease or disorder, in particular, cancer or a condition or symptom associated therewith. In addition, a synergistic effect can result in improved efficacy of therapies in the prevention, management, or treatment of a disease or disorder, in particular, cancer or a condition or symptom associated therewith. Finally, the synergistic effect of a combination of therapies may avoid or reduce adverse or unwanted side effects associated with the use of any single therapy.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects can refer specifically to an increase in uterine cell proliferation, as well as to an increase in the frequency of uterine cancer and an increase in the risk of developing uterine cancer. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56^{th} ed., 2002).

“Without attendant risk in increase of uterine cancer” refers to a lowered or eliminated risk of developing uterine cancer as compared to patients who have an increased risk for developing uterine cancer due to a course of anti-estrogen therapy.

By “therapeutically effective amount or dose” or “therapeutically sufficient amount or dose” or “effective or sufficient amount or dose” herein is meant a dose that produces therapeutic effects for which it is administered, in the context of the combination therapy in which it is administered. Often, the therapeutically effective or sufficient amount or dose of the compounds comprising the pharmaceutical compositions of the invention will be lower when administered in the specific combinations, than the doses that would be therapeutically effective or sufficient when the compounds are administered separately. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington. The Science and Practice of Pharmacy, 20th Edition, 2003, Gennaro, Ed., Lippincott, Williams & Wilkins). In some embodiments, a therapeutically effective amount refers to that amount of the therapeutic agent sufficient to destroy, modify, control or remove primary, regional or metastatic cancer tissue. A therapeutically effective amount may refer to the amount of therapeutic agent sufficient to delay or minimize the spread of cancer. A therapeutically effective amount may also refer to the amount of the therapeutic agent that provides a therapeutic benefit in the treatment or management of cancer. Further, a therapeutically effective amount with respect to a therapeutic agent of the invention means that amount of therapeutic agent alone, or in combination with other therapies, that provides a therapeutic benefit in the treatment or management of cancer. In sensitized cells, the therapeutically effective dose can often be lower than the conventional therapeutically effective dose for non-sensitized cells. In some embodiments, a therapeutically effective amount refers to the amount of a therapeutic agent that, e.g., reduces the proliferation of cancer cells, increases the death of cancer cells or, reduces the size of a tumor or spread of a tumor in a subject. Preferably, a therapeutically effective amount of a therapeutic agent reduces the size of a tumor or the spread of a tumor in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. In some embodiments, a therapeutically effective amount refers to the amount of a therapeutic agent that increases survival by 1 month, 2 months, 6 months, 1 year, 2 years, 3 years, 4 years, 5 years, 6 years, 7 years, 8 years, 9 years, 10 years, or more. In some embodiments, a therapeutically effective amount refers to the amount of a therapeutic agent that prevents the progression from DCIS or atypical hyperplasia to breast cancer.

The HDAC inhibitors encompassed by the methods and compositions of the present invention can be any known to those of skill in the art, particularly those shown in FIGS. 8A-8F, and described in Minucci et al, Nature 6:38-51 (2006). According to the invention, in some preferred embodiments the HDAC inhibitor is carbamazepine, and in other embodiments the HDAC inhibitor is valproic acid. In yet other embodiments, the HDAC inhibitor may be TSA, SAHA, VPA derivatives, MS-275, clyclic hydroxamic acid-containing peptide, Apicidin, Trapoxin, or other HDAC inhibitors known by the skilled practitioner to be effective. Other HDAC inhibitors encompassed by the methods and compositions of the invention include the VPA derivatives as described in U.S. Patent Application Nos. 20050038113 to Groner, and 20040087652 to Gottlicher, as well as the compounds used to inhibit HDAC as disclosed in U.S. Patent Application Nos. 20070135438 to Payne, 20070060614 and 20070190022 to Bacopoulos, 20050107348 to Lan-Hargest, and 20070037738 to Hentsch, as well as U.S. Pat. Nos. 7,169,801, 6,110,955, 6,905,669, and 7,126,001. Other HDAC inhibitors encompassed by the methods and compositions of the invention include the sulfonyl derivatives as described in U.S. Pat. No. 7,205,304 to Van Emelen, the alpha-ketoepoxide compounds of U.S. Pat. No. 7,057,057 to Lan-Hargest, the HDAC inhibitors based on trihalomethylcarbonyl compounds as described in U.S. Pat. No. 7,193,105 to Lan-Hargest, and the HDAC inhibitors based on alpha-chalcogenmethylcarbonyl compounds of U.S. Pat. No. 7,214,831 to Lan-Hargest. Each of the above patents and patent application publications is hereby incorporated by reference in its entirety.

In one aspect, the present invention provides a method of treating and methods of preventing estrogen receptor positive breast cancer, comprising administering to a subject a therapeutically effective amount of an HDAC inhibitor in combination with a course of hormonal therapy. Various combinations of HDAC inhibitors and hormonal therapies are contemplated as useful in treating estrogen receptor positive breast cancer.

In some embodiments, particularly in combination with hormonal therapies, the HDAC inhibitor is not valproic acid and in some embodiments the HDAC inhibitor is not SAHA. In other embodiments of the invention, particularly when in combination with hormonal therapies, and more particularly tamoxifen, the HDAC inhibitor is not carbamazepine.

In one embodiment, the daily dose of valproic acid is from about 15/mg/kg to about 60 mg/kg.

In one embodiment, the dose of valproic acid is sufficient to achieve from about 300 to about 1000 micromolar in patient serum. In another embodiment, the dose of valproic acid is sufficient to achieve from about 300 to about 867 micromolar in patient serum. In another embodiment, the dose of valproic acid is sufficient to achieve from about 500 to about 1000 micromolar in patient serum.

In some embodiments, the dose of carbamazepine is from about 800 mg/day to about 1600 mg/day. In other embodiments, the dose of carbamazepine is from about 800 mg/day to about 1200 mg/day. In yet other embodiments, the dose of carbamazepine is from about 200 mg/day to about 600 mg/day.

In some embodiments, the dose of SAHA is from about 200 mg/day to about 600 mg/day. In another embodiment, the dose of SAHA is about 400 mg/day.

Hormonal agents are a group of drugs that regulate the growth and development of their target organs. Most of the hormonal agents used in the treatment of breast cancer are sex steroids and their derivatives and analogs thereof, such as estrogens, androgens, and progestins. These hormonal agents may serve as antagonists of receptors for the sex steroids to down regulate receptor expression and transcription of genes. Such hormonal therapy agent include, but are not limited to synthetic estrogens (e.g. diethylstibestrol), antiestrogens (e.g. tamoxifen, fulvestrant, fluoxymesterol, raloxifene, and torimefene), antiandrogens (bicalutamide, nilutamide, flutamide), aromatase inhibitors (e.g., aminoglutethimide, anastrozole, letrozole, and tetrazole), ketoconazole, goserelin acetate, leuprolide, megestrol acetate and mifepristone.

In some preferred embodiments, the hormonal therapy is anti-estrogen therapy, which can be, but is not limited to tamoxifen, raloxifene, fulvestrant, and torimefene. In other preferred embodiments, the hormonal therapy can be estrogen ablation therapy, including an aromatase inhibitor. According to the invention, the aromatase inhibitor can be, but is not limited to exemestane, letrozole, fadrozole, retrozole, and anastrozole. Any form of hormonal therapy known to one of skill in the art for the treatment of breast cancer is contemplated as useful in the combination therapies of the present invention.

In some embodiments, the dose of tamoxifen is from about 10 mg/day to about 50 mg/day. In another embodiment, the dose of tamoxifen is about 20 mg/day. In one embodiment in which a patient has metastatic breast cancer, the dose of tamoxifen is from about 20 mg/day to about 40 mg/day.

In one embodiment, the dose of letrozole is from about 1 mg/day to about 5 mg/day. In another embodiment, the dose of letrozole is about 2.5 mg/day.

In one embodiment, the dose of exemestane is from about 10 mg/day to about 40 mg/day. In another embodiment, the dose of exemestane is about 25 mg/day.

In yet another embodiment, the dose of anastrozole is from about 0.5 mg/day to about 3 mg/day. In another embodiment, the dose of anastrozole is about 1 mg/day.

In some embodiments, compounds are administered in combination, with ratios of those compounds which preserve the recommended daily doses of the compounds. In some embodiments, compounds are administered in combination, with ratios of those compounds which preserve the ranges of doses as described herein.

In one embodiment, the ratio of tamoxifen to VPA is 1 part tamoxifen to 45-180 parts VPA, for a 60 kg patient.

In another embodiment, where the patient has metastatic breast cancer, the ratio of tamoxifen to VPA is 1 part tamoxifen to 22.5-180 parts VPA, for a 60 kg patient.

When the combination comprises administering tamoxifen or raloxifene along with an HDAC inhibitor, there is no attendant increase in the risk of uterine cancer. Treatment with tamoxifen or raloxifene is compromised by an increased risk in uterine cancer. A distinct advantage of the present invention is that administration of an HDAC inhibitor in combination with tamoxifen or raloxifene reduces or eliminates the risk of uterine cancer. In some embodiments, this risk is reduced by 10%, 20%, 30%, 40%, 50%, 60%, 70%, 80%, 90%, 95%, or it is virtually or wholly eliminated.

The present invention also encompasses methods of treating and methods of preventing breast cancer comprising administering to a subject suffering therefrom a therapeutically effective amount of an HDAC inhibitor in combination with a therapeutically effective amount of one or more of an IGF-1 receptor inhibitor, an EGFR inhibitor, or an mTOR inhibitor. In some embodiments, the HDAC inhibitor is not VPA when the HDAC inhibitor is in combination with an EGFR inhibitor.

In a further aspect of the invention, methods are provided for treating and for preventing breast cancer, comprising administering to a subject suffering therefrom a therapeutically effective amount of an HDAC inhibitor in combination with a course of hormonal therapy as well as an additional active ingredient that is effective to treat breast cancer in the combination. In some embodiments, the method comprises administering a combination comprising an HDAC inhibitor, a course of hormonal therapy, and one or more of an IGF-1 receptor inhibitor, an EGFR inhibitor, or an mTOR inhibitor.

In yet another aspect of the invention, methods are provided for treating and for preventing breast cancer, comprising administering to a subject suffering therefrom a therapeutically effective amount of a course of hormonal therapy in combination with one or more of an IGF-1 receptor inhibitor, an EGFR inhibitor, or an mTOR inhibitor.

In yet another aspect of the invention, methods are provided for treating and for preventing breast cancer, comprising administering to a subject suffering therefrom a therapeutically effective amount a combination of two or more of an IGF-1 receptor inhibitor, an EGFR inhibitor, or an mTOR inhibitor.

In one embodiment, the combination comprises VPA and tamoxifen. In another embodiment, the combination comprises TSA and tamoxifen. In another embodiment, the combination comprises SAHA and tamoxifen. In yet another embodiment, the combination comprises carbamazepine and tamoxifen.

The IGF-1R inhibitors contemplated in the methods and compositions of the present invention can be any known to one of skill in the art. The IGF-1R inhibitor may be for example, picropodophyllin, podophyllotoxin, podophyllotoxin derivatives, including those disclosed in U.S. Patent Application No. 20070123491 to Axelson, EGCG, cyclolignans such as those disclosed in U.S. Patent Application No. 2004/0186169 to Larsson, and IGF-1R inhibitors such as those disclosed in U.S. Patent Application No. 20060193772 to Ochiai. Each of the above patent applications is hereby incorporated by reference in its entirety.

The EGFR inhibitors contemplated in the methods and compositions of the present invention can be any known to one of skill in the art. The EGFR inhibitor may be for example, gefitinib, erlotinib, cetuximab, imatinib, genistein, genistin, quercetin, equol, staurosporine, aeroplysinin, indocarbazole, lavendustin, piceatannol, kaempferol, daidzein, erbstatin, and tyrphostins.

The mTOR inhibitors contemplated in the methods and compositions of the present invention can be any known to one of skill in the art. The mTOR inhibitor may be for example, temsirolimus, everolimus, rapamycin and rapamycin derivatives, including those rapamycin derivatives disclosed in U.S. Patent Application No. 20040147541 to Lane, which is hereby incorporated by reference in its entirety.

In one embodiment, the combination comprises tamoxifen, carbamazepine, and picropodophyllin. In another embodiment, the combination comprises tamoxifen, carbamazepine, and rapamycin. In yet another embodiment, the combination comprises tamoxifen, carbamazepine, picropodophyllin, and rapamycin.

In one embodiment, the combination comprises carbamazepine and picropodophyllin. In another embodiment, the combination comprises carbamazepine and rapamycin. In yet another embodiment, the combination comprises carbamazepine, picropodophyllin, and rapamycin.

In one embodiment, the combination comprises tamoxifen and picropodophyllin. In another embodiment, the combination comprises carbamazpeine and rapamycin.

In one embodiment, the combination comprises tamoxifen, VPA, and picropodophyllin. In another embodiment, the combination comprises tamoxifen, VPA, and rapamycin. In yet another embodiment, the combination comprises tamoxifen, VPA, picropodophyllin, and rapamycin.

In one embodiment, the combination comprises VPA and picropodophyllin. In another embodiment, the combination comprises VPA and rapamycin. In yet another embodiment, the combination comprises VPA, picropodophyllin, and rapamycin.

In one embodiment, the combination comprises tamoxifen and picropodophyllin. In another embodiment, the combination comprises VPA and rapamycin.

In one embodiment, the combination comprises tamoxifen, VPA, and gefitinib. In another embodiment, the combination comprises tamoxifen, VPA, rapamycin, and gefitinib.

In one embodiment, the combination comprises rapamycin and gefitinib.

In one embodiment, the combination comprises tamoxifen, carbamazepine, and gefitinib. In another embodiment, the combination comprises tamoxifen, carbamazepine, rapamycin, and gefitinib.

In one embodiment, the combination comprises tamoxifen, carbamazepine, and EGCG. In another embodiment, the combination comprises tamoxifen, EGCG, and rapamycin. In yet another embodiment, the combination comprises tamoxifen, carbamazepine, EGCG, and rapamycin. In an additional embodiment, the combination comprises tamoxifen and EGCG. In another embodiment, the combination comprises carbamazepine and EGCG. In yet another embodiment, the combination comprises carbamazepine, EGCG, and rapamycin.

In one embodiment, the combination comprises tamoxifen, VPA, and EGCG. In another embodiment, the combination comprises tamoxifen, VPA, EGCG, and rapamycin. In another embodiment, the combination comprises VPA and EGCG. In yet another embodiment, the combination comprises VPA, EGCG, and rapamycin.

In one embodiment, the dose of rapamycin is from about 0.125 mg/day to about 1 mg/day.

In one embodiment, the dose of gefitinib is from about 200 mg/day to about 300 mg/day. In another embodiment, the dose of gefitinib is about 250 mg/day.

In one embodiment, the dose of erlotinib is from about 100 mg/day to about 150 mg/day.

As relates to the inhibitors described herein, many different procedures can be used to specifically inactivate or silence a target gene or inhibit the activity of its gene product, as encompassed by the present invention. Inhibition of protein activity can be brought about at the level of gene transcription, protein translation or post-translational modifications. For instance, the activity of a protein can be inhibited by directly inhibiting the activity of the protein such as altering a catalytic domain or alternatively by reducing the amount of the protein in the cell by reducing the amount of mRNA encoding the protein. In each case, the level of protein activity in the cell is reduced. Various techniques can be used to knock down the activity of a protein and these include knockout technologies (antibodies, antisense RNA, and RNA interference) and compounds that specifically inhibit the protein activity.

In certain embodiments, an RNA interference (RNAi) molecule is used to decrease expression of a gene. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of the gene of interest to produce a phenotype that is the same as that of a null mutant of the gene of interest (Wianny & Zernicka-Goetz, 2000, Nature Cell Biology 2: 70-75).

Any therapy (e.g., chemotherapies, radiation therapies, hormonal therapies, and/or biological therapies/immunotherapies) which is known to be useful, or which has been used or is currently being used for the prevention, treatment, management or amelioration of cancer or one or more symptoms thereof can be used in accordance with the invention, and may be combined with any of the compositions described herein, and may encompass the other active ingredient described for some of the combination therapies herein.

In some embodiments, the anti-cancer agents contemplated in the methods and compositions of the present invention, which can be administered in combination with the compositions of the present invention include, but are not limited to doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), herceptin, tamoxifen, the combination of tamoxifen and cytotoxic chemotherapy, taxanes (such as docetaxel and paclitaxel). In a further embodiment, the combinations of the invention can be administered with taxanes plus standard doxorubicin and cyclophosphamide for adjuvant treatment of node-positive, localized breast cancer.

In one embodiment, the dose of doxorubicin hydrochloride (i.v.) is 60-75 mg/m² on day 1 of treatment.

In another embodiment, the dose of epirubicin (i.v.) is 100-120 mg/m² on day 1 of each cycle or divided equally and given on days 1-8 of the treatment cycle.

In yet another embodiment, the dose of docetaxel (i.v.) is 60-100 mg/m² over 1 hour.

In another embodiment, the dose of paclitaxel (i.v.) is 175 mg/m² over 3 hours.

It is within the scope of the present invention to treat many different types of subjects or patients, though preferably, the subject is a mammal. Preferred mammals include primates such as humans and chimpanzees, domestic animals such, as horses, cows, pigs, etc. and pets such as dogs and cats. Most preferably, the invention encompasses treating humans, and in particular, human females. The pharmaceutical compositions described herein may be used for the treatment of cancer, particularly for breast cancer. The pharmaceutical compositions and methods of the present invention can be used to treat an individual with any type and/or stage of breast cancer. There are several types of breast cancer and there are several stages of breast cancer, all of which are contemplated as treated by the methods and compositions of the present invention.

The present invention can be used to treat a patient with any type of breast cancer. Breast cancers may include carcinoma in situ, infiltrating (or invasive) ductal carcinoma, infiltrating (or invasive) lobular carcinoma, medullary carcinoma, colloid carcinoma, tubular carcinoma, and inflammatory carcinoma.

In addition to the different types of breast cancer, there are also different stages of breast cancer, referred to as stages 0-IV. The system most often used to describe the growth and spread of breast cancer is the TNM staging system, also known as the American Joint Committee on Cancer (AJCC) system. In TNM staging, information about the tumor, nearby lymph nodes, and distant organ metastases is combined and a stage is assigned to specific TNM groupings. The grouped stages are described using Roman numerals from I to IV. The clinical stage is determined by results from physical examination and tests. The pathologic stage includes the findings of the pathologist after surgery. Most of the time, pathologic stage is the most important stage because usually the cancer isn't known to have spread to lymph nodes until the pathologist examines them under the microscope. In the TNM staging system, T stands for the size of the cancer (measured in centimeters; 2.54 centimeters 1 inch); N stands for spread to lymph nodes in the area of the breast, and M is for metastasis (spread to distant organs of the body).

The T category describes the original (primary) tumor. Tis: Tis is used only for carcinoma in situ or noninvasive breast cancer such as ductal carcinoma in situ, (DCIS) or lobular carcinoma in situ (LCIS). T1: The cancer is 2 cm in diameter (about ¾ inch) or smaller. T2: The cancer is more than 2 cm but not more than 5 cm in diameter. T3: The cancer is more than 5 cm in diameter. T4: The cancer is any size and has spread to the chest wall, the skin, or lymphatics.

The N category is based on which of the lymph nodes near the breast, if any, are affected by the cancer. N0: The cancer has not spread to lymph nodes. N1: The cancer has spread to lymph nodes under the arm on the same side as the breast cancer. Lymph nodes have not yet attached to one another or to the surrounding tissue. N2: The cancer has spread to lymph nodes under the arm on the same side as the breast cancer and are attached to one another or to the surrounding tissue or enlarged. Or, the cancer can be seen to have spread to the internal mammary lymph nodes (next to the sternum), but not to the lymph nodes under the arm. N3: The cancer has spread to lymph nodes above or just below the collarbone on the same side as the cancer, and may or may not have spread to lymph nodes under the arm. Or, the cancer has spread to internal mammary lymph nodes and lymph nodes under the arm, both on the same side as the cancer.

M categories: The M category depends on whether the cancer has spread to any distant tissues and organs. M0: No distant cancer spread. M1: Cancer has spread to distant organs.

There are different types of staging. Clinical staging estimates how much cancer there is based on the results of the physical exam, imaging tests x-rays, CT scans, etc.) and sometimes biopsies of affected areas. For certain cancers the results of other tests, such as blood tests, are also used in staging. Pathologic staging can only be done on patients who have had surgery to remove or explore the extent of the cancer. It combines the results of clinical staging (physical exam, imaging tests, etc.) with the results from the surgery. In some cases, the pathologic stage may be different from the clinical stage (for example, if the surgery shows the cancer is more extensive than it was previously thought to be). Restaging is sometimes used to determine the extent of the disease if a cancer recurs (comes back) after treatment.

In one embodiment, the methods and compositions of the present invention are used to treat patients with stage I breast cancer.

In one embodiment, the methods and compositions of the present invention are used to treat patients with stage II breast cancer.

In one embodiment, the methods and compositions of the present invention are used to treat patients with stage III breast cancer.

In one embodiment, the methods and compositions of the present invention are used to treat patients with stage IV breast cancer, i.e. patients with metastatic cancer.

In another embodiment, the patient having breast cancer has already failed other treatment regimens such as chemotherapy.

In one embodiment, the methods and pharmaceutical compositions of the present invention may be used to prevent the development of a cancer, particularly in an individual at higher risk than average to develop such cancer than other individuals, or to treat a patient afflicted with breast cancer.

There are a number of ways to assess an individual's risk for breast cancer, and any means of risk assessment is contemplated by the present invention as determining which subjects are at risk for breast cancer and can undergo treatment via the methods and compositions of the present invention. The invention contemplates treatment for individuals with a higher than average lifetime risk for breast cancer, the average being about one in eight women in the U.S.

The invention provides methods treating asymptomatic patients who have a likelihood of benefiting from therapeutic treatment of breast cancer. The asymptomatic patients can comprise patients in any of the many high risk groups for breast cancer. The high risk groups can include e.g. patients with a family history of breast cancer, patients of increasing age (e.g, patients 40 years of age or older), menopausal patients, patients having at least one high risk parity factor (e.g. early start of menses, late onset of menopause, no pregnancies, or late-age pregnancy), patients having high risk gene status (e.g. patients testing positive for a mutation in BRCA1 or BRCA2 genes, or others, as described below), patients having at least one previous breast biopsy (benign or otherwise), patients having a previous diagnosis of breast cancer, and patients having any other risk factor for breast cancer. Other risk factors are continually being defined and can include such considerations, as geographic location (e.g. where women living in a particular region have been found to have a higher incidence of breast cancer). Diet is also thought to play a role in breast cancer risk; specifically women who include more fat in their diet may be more likely to develop breast cancer (see Kniget et al. Cancer Epidemiol Biomarkers Prev 8(2):123-8, 1999).

The Gail model is a common means of determining risk for breast cancer, and was developed based on the Breast Cancer Detection Demonstration Project (see Gail, M. et al, J Natl Cancer Inst., 1989. 81: p. 1879-86). The risk factors used in the Gail model are age, age at menarche, age at first live birth, number of previous breast biopsies, number of first-degree relatives with breast cancer. These risk factors are broadly consistent with those selected from other large population-based studies. A revised Gail model also incorporates race, presence of atypical hyperplasia on breast biopsy, and 1987 population rates of breast cancer and death from other causes.

Another commonly used prediction model is the Claus model, based on the Cancer and Steroid Hormone Study (see Claus E. et al., Cancer, 1994. 73: 643-51) and incorporates more extensive information about family history. The Claus model provides individual estimates of breast-cancer risk according to decade from 29-79 years of age. It takes into account factors such as the number of first-degree and number of second-degree relatives with breast cancer, as well as different combinations of different degree relatives with breast cancer.

The invention also contemplates treatment for early stages of cancer, for recurrent cancer, and for those in remission from cancer.

The present invention also encompasses treatment for subjects with markers for breast cancer, including, but not limited to having mutations or other alterations in the genes, BRCA1, BRCA2, P53, P65, ATM, or pS2, or a changed ratio of the expression of the genes HOXB13 and IL17BR, amplification of the AIB1/pCIP coactivator gene, overproduction of HER2 protein and/or gene, and alterations in levels of hormones, such as estrogen and progesterone, or their receptors.

Markers can also include neoplastic ductal epithelial cells, transforming growth factor-β, carcinoma embryonic antigen (CEA), prostate specific antigen (PSA), Erb B2 antigen, gross cystic disease fluid protein-15 (GCDFP-15), lactose dehydrogenase (LDH), measured in the ductal fluid, or a chromosomal abnormality in the ductal epithelial cells. Where the marker is neoplastic ductal epithelial cells, the cells can be at a stage including hyperplasia, atypical hyperplasia, low grade ductal carcinoma in situ (LG-DCIS), high grade ductal carcinoma in situ (HG-DCIS) or invasive carcinoma. The present invention encompasses providing the pharmaceutical compositions described herein to treat subjects with any of the described markers, and also to prevent the progression from DCIS and from atypical hyperplasia to breast cancer.

The combinations of the invention may be administered either prior to or following surgical removal of primary tumors and/or treatment such as administration of radiotherapy or conventional chemotherapeutic drugs.

The methods and compositions of the present invention may be used advantageously in combination with any other treatment regimen for breast cancer. Treatments for breast cancer are well known in the art and continue to be developed. Treatments include but are not limited to surgery, including axillary dissection, sentinel lymph node biopsy, reconstructive surgery, surgery to relieve symptoms of advanced cancer, lumpectomy (also called breast conservation therapy), partial (segmental) mastectomy, simple or total mastectomy, modified radical mastectomy, and radical mastectomy; immunotherapy, e.g. using Herceptin™ (trastuzumab), an anti-HER2 humanized monoclonal antibody developed to block the HER2 receptor; bone marrow transplantation; peripheral blood stem cell therapy; bisphosphonates; additional chemotherapy agents; radiation therapy; acupressure; and acupuncture. Any combination of therapies may be used in conjunction with the present invention.

The methods and compositions comprising the combination therapies described herein may also be used to reduce the proliferation of cancer cells, increase the death of cancer cells or, reduces the size of a tumor or spread of a tumor in a subject. It is contemplated by the present invention that the combination therapies described herein may reduce the size of a tumor or the spread of a tumor in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. In some embodiments, the combination therapies described herein may increase survival by 1 month, 2 months, 6 months, 1 year, 2 years, 3 years, 4 years, 5 years, 6 years, 7 years, 8 years, 9 years, 10 years, or more, it may render the subject disease-free, or it may prevent the progression from DCIS or atypical hyperplasia to breast cancer.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003).

The compounds of the invention may be formulated into pharmaceutical compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopoeia Convention, Inc., Rockville, Md. (1989), the relevant contents of which is incorporated herein by reference.

In a preferred embodiment, a composition of the invention is a pharmaceutical composition. Such compositions comprise a prophylactically or therapeutically effective amount of one or more prophylactic or therapeutic agents, including at least one HDAC inhibitor and at least one hormonal therapy agent and a pharmaceutically acceptable carrier. In other embodiments, such compositions comprise a prophylactically or therapeutically effective amount of at least one HDAC inhibitor, and a prophylactically or therapeutically effective amount of one or more of an IGF-1R inhibitor, an EGFR inhibitor, or an mTOR inhibitor, and optionally a prophylactically or therapeutically effective amount of one or more hormonal therapy agents and a pharmaceutically acceptable carrier. In yet other embodiments, such compositions comprise a prophylactically or therapeutically effective amount of one or more prophylactic or therapeutic agents, including at least one HDAC inhibitor, at least one hormonal therapy agent, an additional active ingredient as described herein, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering one or more prophylactic or therapeutic agents, care must be taken to use materials to which the prophylactic or therapeutic agents do not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, pp. 317-327; see generally above).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Publication No. WO 99/15154; and International Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938, International publication No. WO 91/05548, International publication No. WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is one or more nucleic acid molecules encoding one or more prophylactic or therapeutic agents, the nucleic acid can be administered in vivo to promote expression of its encoded prophylactic or therapeutic agents, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of suitable routes of administration include, but are not limited to, parenteral (e.g., intravenous, intramuscular, intradermal, intra-tumoral, intra-synovial, and subcutaneous), oral (e.g., inhalation), intranasal, transdermal (topical), transmucosal, intra-tumoral, intra-synovial, vaginal, and rectal administration. In a specific embodiment, the composition is formulated in accordance with routine procedures as a pharmaceutical composition adapted for intravenous, subcutaneous, intramuscular, oral, intra-tumoral, intra synnovial, intranasal or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the compositions of the invention are to be administered topically, the compositions can be formulated in the form of, e.g., a toothpaste, ointment, cream, transdermal patch, lotion, gel, oral gel, shampoo, spray, aerosol, solution, emulsion, or other form well-known to one of skill in the art. See, e.g., Remington's Pharmaceutical Sciences and Introduction to Pharmaceutical Dosage Forms, 4.sup.th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., gum, tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Kits

The invention provides a pharmaceutical pack or kit comprising one or more containers filled with individual components (in pharmaceutical formulations) of the combination therapies described herein; for example, contained filled with an HDAC inhibitor and one or more hormonal therapy agents, and/or one or more therapeutic or prophylactic agents such as an IGF-1R inhibitor, an EGFR inhibitor, an mTOR inhibitor, or another active ingredient. Containers may also be filled with an HDAC inhibitor, and one or more therapeutic or prophylactic agents such as an IGF-1R inhibitor, an EGFR inhibitor, an mTOR inhibitor, and/or another active ingredient. The pharmaceutical pack or kit may further comprises one or more other prophylactic or therapeutic agents useful for the treatment of a disease or disorder. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides pharmaceutical packs or kits that can be used in the above methods. In one embodiment, a kit comprises at least one HDAC inhibitor and at least one hormonal therapy agent in one or more containers. The kit may further comprises one or more other prophylactic or therapeutic agents, or active ingredients useful for the treatment of cancer in one or more containers. In other embodiments, the kit may comprise at least one HDAC inhibitor, and one or more of at least one or more of an IGF-1R inhibitor, an EGFR inhibitor or mTOR inhibitor. Examples of such agents and compounds are disclosed above.

Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intratumoral, intra-synovial, topical or mucosal delivery.

In a specific embodiment, the unit dosage form is suitable for intravenous, intramuscular, intratumoral, intra-synovial, or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, calcium concentration, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material.

In a specific embodiment, an article of manufacture comprises packaging material and a pharmaceutical agent and instructions contained within said packaging material, wherein said pharmaceutical agent comprises at least one HDAC inhibitor, at least one hormonal therapy agent, optionally another “active ingredient,” and a pharmaceutically acceptable carrier, and said instructions indicate a dosing regimen for preventing, treating or managing a subject with cancer. In another embodiment, an article of manufacture comprises packaging material and a pharmaceutical agent and instructions contained within said packaging material, wherein said pharmaceutical agent comprises an HDAC inhibitor, and one or more of at an IGF-1R inhibitor, EGFR inhibitor, or mTOR inhibitor, and a pharmaceutically acceptable carrier, and said instructions indicate a dosing regimen for preventing, treating or managing a subject with a cancer.

In therapeutic use for the treatment of cancer, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician.

Characterization and Demonstration of Therapeutic or Prophylactic Utility

Toxicity and efficacy of the prophylactic and/or therapeutic treatments and protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In order to determine therapeutic or prophylactic utility, it is encompassed by the present invention to use any of the assays described herein, including those described and illustrated in the Examples section below, as well as those known in the art. Also encompassed by the invention to determine therapeutic or prophylactic utility are any relevant cancer, and more specifically, breast cancer animal models. For example, one may utilize a an MCF-7 xenograft model, or a modified MCF-7 xenograft model (Hale L. V. et al., 1997, Lab Anim Sci., 47(1):82-85). Further encompassed by the invention, pending safety and efficacy, are clinical trials to assess the combinations of the present invention.

### EXAMPLES

It is understood that the following examples and embodiments described herein are for illustrative purposes only and that various modifications or changes in light thereof will be suggestive to persons skilled in the art and are to be included within the spirit and purview of this application and the scope of the appended claims. All publications, patents, and patent applications cited herein are hereby incorporated by reference in their entirety for all purposes.

Unless otherwise specified, in the examples below, methods were carried out in the following way.

Cell Culture and Ligands. MCF-7 cells were provided by C. Walker (MD Anderson Cancer Center, Houston, Tex.) and were routinely cultured in IMEM (Invitrogen, Grand Island, N.Y.) with 10% fetal calf serum (FBS) (Hyclone, Logan, Utah). Ishikawa cells were also provided by C. Walker and cultured in DMEM/Ham's F12 with 10% FBS. T47-D cells were obtained from ATCC and were routinely cultured in DMBM (Invitrogen) plus 10% FBS. ZR-75-1 cells were provided by B. Hahn (University of California, San Francisco) and were routinely cultured in RPMI (Invitrogen) plus 10% PBS. MCF-7/neo and MCF-7/HER2 (clone 18) cells were also obtained from B. Hahn and cultured in DMEM plus 10% FBS. MCF-7aro cells were provided by S. Chen (Beckham Research Institute of the City of Hope) and routinely grown in DMEM/Ham's F12 plus 10% FBS. For all experiments cells were switched to phenol red-free media containing 5% charcoal/dextran-stripped FBS (Hyclone) for 3-5 days prior to start of the experiment. Cells were treated with ligands in media containing 2-5% stripped PBS for the indicated times. 17β-estradiol (E2), 4-trans-hydroxytamoxifen (OH-Tam), raloxifene, trichostatin A, valproic acid sodium salt (sodium 2-propylpentanoate) were obtained from Sigma-Aldrich (St. Louis, Mo.). Fulvestrant (ICI 182,780) was obtained from Tocris (Ellisvllle, Mo.) and suberoylanilide hydroxamic acid (SAHA) from BioVision (Mountain View, Calif.).

Cell Proliferation Assays. Cells growing in 24-well dishes were treated with ligands in triplicate for the indicated times. Cells were trypsinized and counted electronically with a Coulter Counter (Coulter Electronics, Hialeah, Fla.). Alternatively, cell proliferation was measured using a fluorescent DNA-binding assay, CyQUANT (Invitrogen) in which cells were treated with ligands in triplicate in 96-well plates for the indicated times and assayed according to the manufacturer's instructions.

Flow cytometry. MCF-7 cells growing in 100 mm dishes were treated with ligands for 48 hours, then trypsinized and fixed in 70% ethanol and stained with 50 mg/ml propidium iodide (Roche, Nutley, N.J.). DNA content was obtained by measuring 10,000 events on a FACScalibur flow cytometer (BD Biosciences, San Jose, Calif.). The percentages of cells in each phase of the cell cycle were determined using the Watson (Pragmatic) model analyzed by FlowJo flow cytometry software (Treestar, San Carlos, Calif.).

Apoptosis. MCF-7 cells growing on coverslips were treated with ligands for 72 hours and unfixed cells were assayed with the Annexin-V-FLOUS Staining kit (Roche), according to manufacturer's instructions. Total cell number was determined by counting nuclei stained with Hoescht 33342 (Roche). Fluorescence was analyzed using a Zeiss Axioplan fluorescent microscope (Zeiss, Thomwood, N.Y.).

Transfections. MCF-7 cells growing in 12-well dishes were transfected with ERE-Luc and β-galactosidase reporter gene using Lipofectamine 2000 transfection reagent (Invitrogen) according to manufacturer's instructions. ERE-Luc reporter gene has been previously described (Liu, M. M., et al., J Biol Chem., 2002. 277(27): p. 24353-60; Webb, P., et al., Mol Endocrinol., 1999. 13(10): p. 1672-85). Five hours after transfection, cells were treated in triplicate with ligands and harvested 24 hours later using a lysis buffer containing 100 MM TrisHCl, 1% Triton-X100 and 1.μ/ml dithiothreitol. Reporter gene activity was measured using assay kits for Luciferase (Promega, Madison, Wis.) and β-galactosidase (Tropix, Bedford, Mass.), according to manufacturer's instructions.

Immunoblotting. MCF-7 cells were grown in 100 mm dishes and treated with ligands in triplicate. Cells were harvested 24 hours later using a lysis buffer containing 65.2 mM Tris-HCl, 154 mM NaC1, 1:100 NP-40, 1:400 sodium doxycholate, 2 mM sodium orthovanadate, 1 mM sodium fluoride, 1 mM phenylmethylsulphonylfluoride, and 1 μg/ml each of leupeptin, aprotinin and pepstatin. Whole cell extracts were separated by SDS-PAGE, transferred to a nitrocellulose membrane, and immunoblotted using standard methods with the following antibodies: ERα, Bik, Bcl-2, and β-tubulin (all from Santa Cruz Biotechnology, Santa Cruz, Calif.) and cyclin D1 (Zymed, San Francisco, Calif.).

Statistical Analysis. All results are presented as mean ±standard error (S.E.M). Statistical significance between treatment groups was determined by ANOVA with Fisher's planned least significant test at p≦0.05 conducted using Statview software (SAS Institute, Cary, N.C.).

### Example 1

### VPA Enhances the Antiproliferative Effect of Tamoxifen

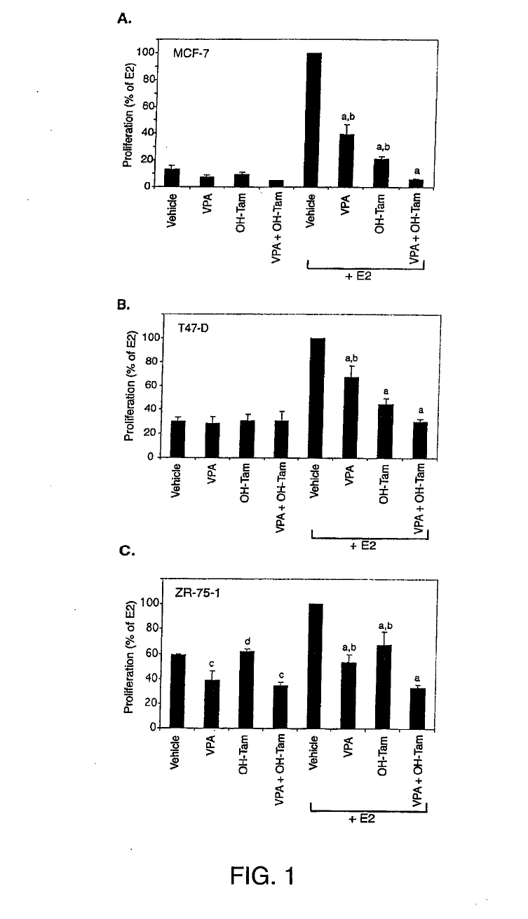

Cellular proliferation was evaluated in three ERα positive breast cancer cell lines after treatment with VPA, tamoxifen, or a combination of both ligands for 6-7 days in vitro. VPA at the therapeutic concentration of 750 μM inhibited MCF 7 cells and in the presence of 17β-estradiol (E2), inhibition by VPA was even more dramatic (FIG. 1A). 10 nM of hydroxytamoxifen, the active metabolite of tamoxifen, inhibited E2-induced proliferation of MCF-7 cells and when combined with VPA, proliferation was inhibited to a greater extent than either ligand alone. T47D and ZR-75-1 cells responded similarly to MCF-7 cells, with VPA and tamoxifen cooperating in their anti-proliferative effects, particularly in the presence of E2 (FIGS. 1B and 1C). ZR-75-1 cells, which exhibited a higher level of basal proliferation compared to the other two cell lines, were also significantly inhibited by co-treatment of VPA and tamoxifen in the absence of E2. Together, these results indicate that VPA and tamoxifen cooperate, combines effectively in their anti-proliferative effects for ER-positive breast cancer cells and suggest an enhanced efficacy over that of either ligand alone.

### Example 2

### VPA Enhances the Potency and Efficacy of Both Antiestrogen and Aromatase Inhibitor Action on Breast Cancer Cells

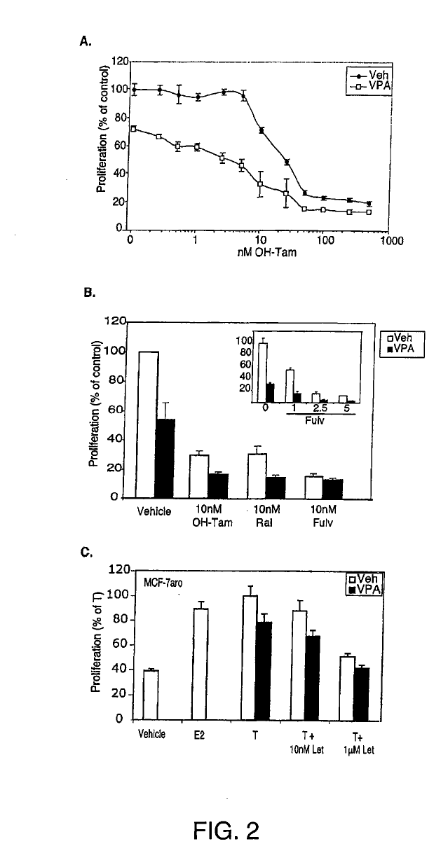

Whether VPA could change the efficacy and/or potency of tamoxifen in a dose responsive proliferation assay was next investigated. MCF-7 cells were treated with a range of concentrations of tamoxifen, both in the presence and absence of 750 μM VPA (FIG. 2A). VPA treatment alone inhibited E2-stimulated cell proliferation by 25% and enhanced the relative efficacy of tamoxifen at all doses tested. VPA also enhanced the IC₅₀ for tamoxifen treatment to 3 nM, compared to 25 nM when tamoxifen was used alone. Thus, VPA enhanced the potency as well as the efficacy of tamoxifen action on cell proliferation.

To determine if VPA could also be effective in enhancing the anti-proliferative activity of other antiestrogens besides tamoxifen, MCF-7 cells were treated with VPA in combination with the selective estrogen receptor modulator raloxifene or the pure antiestrogen fulvestrant (FIG. 2B). Raloxifene substantially decreased E2-stimulated growth and VPA further enhanced its inhibitory effect, similar to that observed with tamoxifen. Higher concentrations of fulvestrant decreased E2-induced proliferation, however, VPA did not further add to its inhibitory effect. Since higher concentrations of fulvestrant increases apoptosis in MCF-7 cells, we also tested sub-saturating doses of fulvestrant, ranging from 1 to 5 nM (Diel, P., K. Smolnikar, and H. Michna, Breast Cancer Res Treat., 1999. 58(2): p. 87-97; Hur, J., et al., Proc Natl Acad Sci USA., 2004. 101(8): p. 2351-6; Somai, S., et al., Int J. Cancer., 2003. 105(5): p. 607-12). VPA enhanced the anti-proliferative effect of lower doses of fulvestrant in a dose responsive manner (FIG. 2C). These data indicate that in addition to tamoxifen, VPA also cooperates, or combines effectively with the anti-proliferative effects of raloxifene and fulvestrant.

Next, MCF-7 cells were used with stably expressed aromatase (MCF-7aro) to determine if VPA would enhance the inhibition of proliferation observed with aromatase inhibitors. After 5 days of treatment, testosterone stimulated proliferation as well as E2, indicating that aromatase is functional in MCF-7aro cells and converting testosterone to E2. The aromatase inhibitor letrozole inhibited testosterone-induced proliferation in a dose responsive manner. VPA inhibited proliferation more than that observed with letrozole alone, regardless of the dose tested. Taken together, these results indicate VPA cooperates, or combines effectively with the antiproliferative effects of the two major forms of hormonal therapy currently used for treating breast cancer, antiestrogens and aromatase inhibitors.

### Example 3

### Other HDAC Inhibitors Behave Similarly to VPA in Enhancing the Actions of Tamoxifen on Breast Cancer Cells

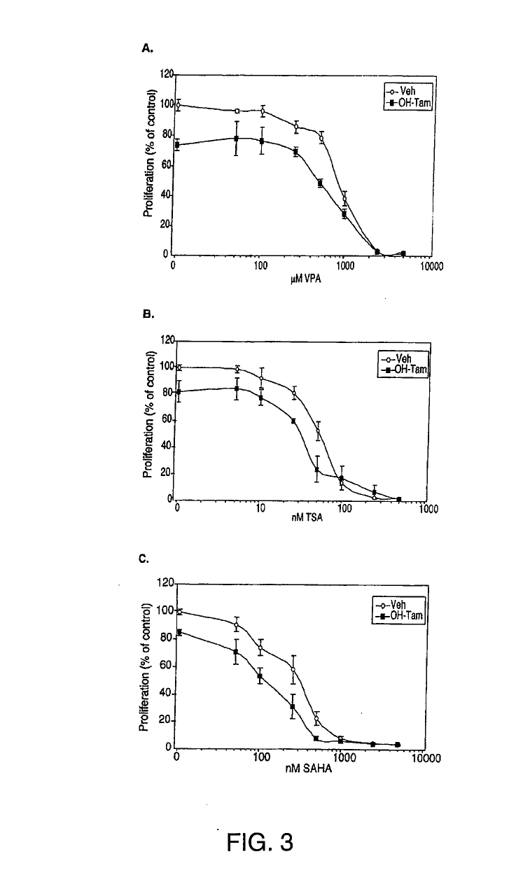

To determine whether tamoxifen may enhance the effectiveness of other HDAC inhibitors, tamoxifen was treated in combination with various doses of TSA and SAHA, two well-described HDAC inhibitors as well as VPA for comparison. Treatment of MCF-7 cells with doses of VPA ranging from 50 μM to 5 M resulted in an IC₅₀ of 800 μM (FIG. 3A). VPA also enhanced the action of tamoxifen, which inhibited cell proliferation about 25% by itself, and combined effectively with VPA at all doses tested. In addition, tamoxifen co-treatment enhanced the potency of VPA, resulting in a slight shift of the IC₅₀ to 500 μM. Both TSA and SAHA, two well-known HDAC inhibitors, had actions similar to that of VPA (FIG. 3B-3C). They enhanced the antiproliferative action of tamoxifen and their IC₅₀ was shifted by the presence of OH-Tam. Thus, the IC₅₀ of TSA alone was 51 nM and co-treatment with tamoxifen shifted the IC₅₀ to 32 nM. Similarly, tamoxifen shifted the IC₅₀ of SAHA from 300 nM to 125 nM. These data suggest that HDAC inhibitors in general cooperate, or combines effectively with the antiproliferative effects of tamoxifen.

### Example 4

### VPA Induces Apoptosis and Enhances the Apoptotic Activity of Tamoxifen

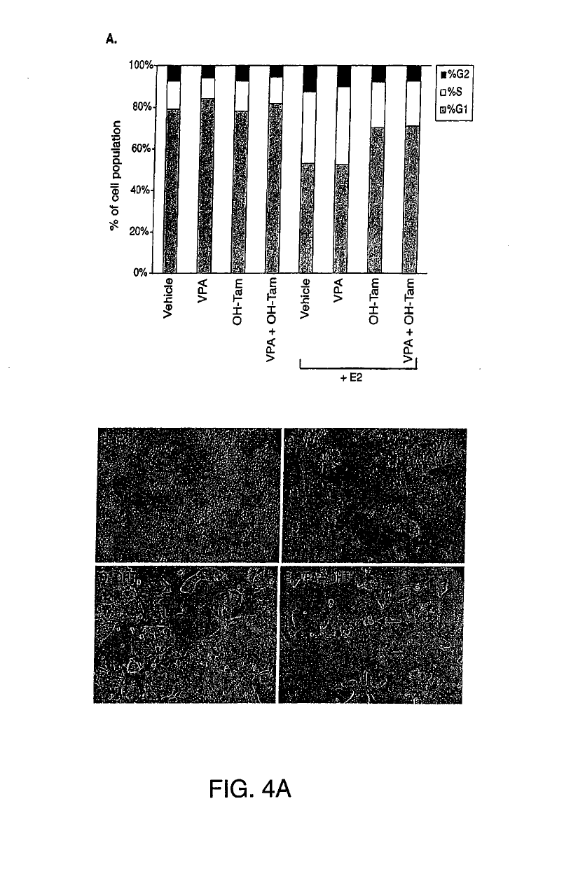

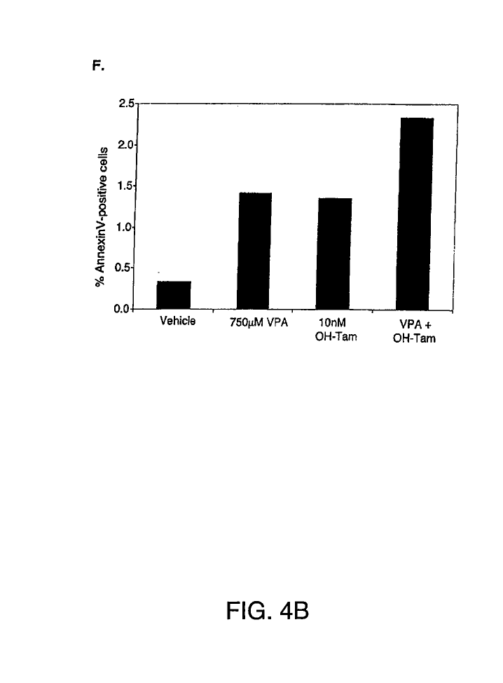

To determine if VPA enhanced the anti-proliferative effect of tamoxifen by halting cell cycle progression, flow cytometry was used to measure the number of cells in each phase of the cell cycle, MCF-7 cells were treated with ligands for 48 hours and the population of cells in G1, S and G2 was estimated based on DNA content (FIG. 4E). VPA treatment alone induced a small arrest in the G1 phase in the absence of E2, but had no effect when E2 was present. Tamoxifen had a dramatic effect of arresting cells in G1 in the presence of E2, as expected. However, the addition of VPA to tamoxifen on E2-induced cell cycle progression did not yield a detectable change, in contrast to the dramatic inhibition of proliferation observed (FIG. 4D and FIG. 1A). These observations suggest that VPA may be enhancing the action of tamoxifen by some means other than altering cell cycle progression, for example by increasing cell death.

Next MCF-7 cells were observed with phase microscopy to determine whether VPA and tamoxifen induced morphological changes such as those observed in cells undergoing apoptosis. After 6 days in culture, E2-treated cells grew into a confluent monolayer, covering virtually every available space in the culture dish (FIG. 4A). VPA-treated cultures exhibited fewer cells as well as an increased number of floating cells (FIG. 4B). Even fewer cells were observed with tamoxifen treatment than for cells treated with VPA (FIG. 4C). VPA and tamoxifen treatment in combination led to a dramatic decrease in cell number, leaving far fewer cells than after treatment with either tamoxifen or VPA alone (FIG. 4D). Additionally, VPA and tamoxifen co-treatment produced an increased proportion of cells exhibiting bright, condensed, and/or rounded cells with an increased number of floating cells, morphologies indicative of cells in late-stage apoptosis.

To further characterize the effect of combination VPA and tamoxifen on apoptosis we employed a quantitative apoptotic assay based on Annexin V staining, which targets disruptions of the phospholipid layer of the membrane from live cells undergoing early-stage apoptosis (Vermes, I., et al., J Immunol Methods., 1995. 184(1): p. 39-51). (FIG. 4F). MCF-7 cells growing for 3 days in the absence of ligands exhibited very low basal levels of AnnexinV positive staining, 0.33% of the total cell population, therefore a large number of cells, at least 2500, were analyzed to obtain an accurate measurement of the apoptotic index. Both VPA and tamoxifen treatments alone induced similar increases in the number of AnnexinV positive cells observed, approximately 1.37%. VPA plus tamoxifen co-treatment further increased the apoptotic index to 2.33%. Since the method used to quantify the apoptotic index excluded floating cells, the number of Annexin V-positive cells may be conservative and the number of apoptotic cells with VPA and/or tamoxifen treatment could in reality be higher. In summary, VPA enhanced tamoxifen-induced apoptosis while having no or little effect on tamoxifen's ability to arrest cell proliferation.

### Example 5

### VPA and Tamoxifen Interactions on Estrogen Receptor Alpha (ERα) Mediated Chances in Gene Expression

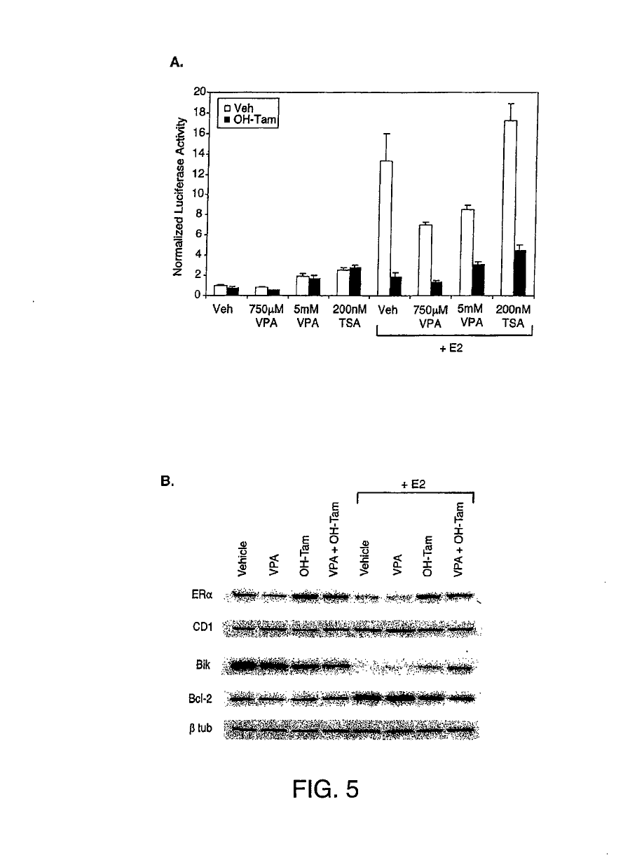

To better understand the effect of VPA on ERα-mediated gene expression, transcriptional activation from a luciferase reporter gene regulated by a consensus estrogen response element (ERE) was evaluated. In MCF-7 cells transiently transfected with the reporter, endogenous ERα stimulated transcription approximately 13-fold with 100 pM E2 treatment. High concentrations of TSA and VPA alone induced transcription and also increased the transcriptional activity of tamoxifen at the ERE. However 750 μM VPA, the dose previously demonstrated to inhibit tamoxifen induced growth, was unable to activate transcription alone or in the presence of tamoxifen (FIG. 5A). Interestingly, although a high dose of VPA alone inhibited E2-induced transcriptional activity, it increased the ability of tamoxifen to stimulate transcription in the presence of E2.

Changes in protein expression from endogenous genes in MCF-7 cells treated for 72 hours with ligands was also evaluated. VPA slightly downregulated ERα protein expression and also attenuated the increased ERα expression that is typically seen with tamoxifen treatment. However, this effect was only observed in the absence of E2. ERα protein levels were undetectable in these lysates and expression was not altered with ligand treatment (data not shown). VPA did not alter cyclin D1 expression nor did it alter tamoxifen-mediated down regulation of cyclin D1, consistent with the lack of effect on cell cycle progression.

The pro-apoptotic gene Bik has been reported to be an essential mediator of apoptosis in MCF-7 cells where its expression is downregulated by E2 and upregulated by estrogen withdrawal or by the pure antiestrogen fulvestrant (Hur, J., et al., Proc Natl Acad Sci USA., 2004. 101(8): p. 2351-6). Under E2-deprived conditions, where Bik protein is expressed at high levels, cotreatment with VPA and tamoxifen had minimal effects on Bik expression (FIG. 5B). In the presence of E2, where Bik protein is down regulated, cotreatment of VPA and tamoxifen combines effectively to upregulate Bik expression better than each ligand alone, using the same conditions in which VPA enhanced the antiproliferative effect of tamoxifen in proliferation assays. The anti-apoptotic protein Bcl-2 was upregulated with E2 treatment and this stimulation was downregulated with tamoxifen. However VPA did not have any effect on Bcl-2 expression either alone or with tamoxifen. In summary, of the proteins analyzed by immunoblotting, Bik protein levels most closely mirrored the increased apoptotic and anti-proliferative effect observed when tamoxifen was co-treated with VPA.

### Example 6

### VPA Enhances the Antiproliferative Effect of Tamoxifen in MCF-7 Cells Overexpressing HER 2/neu

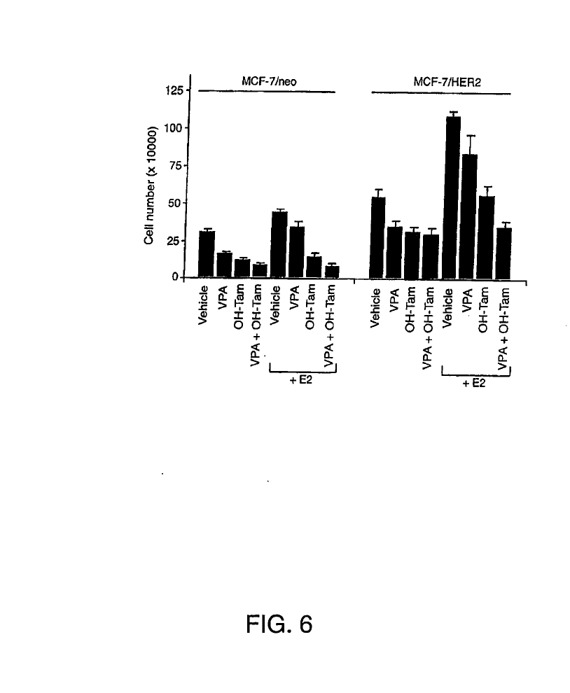

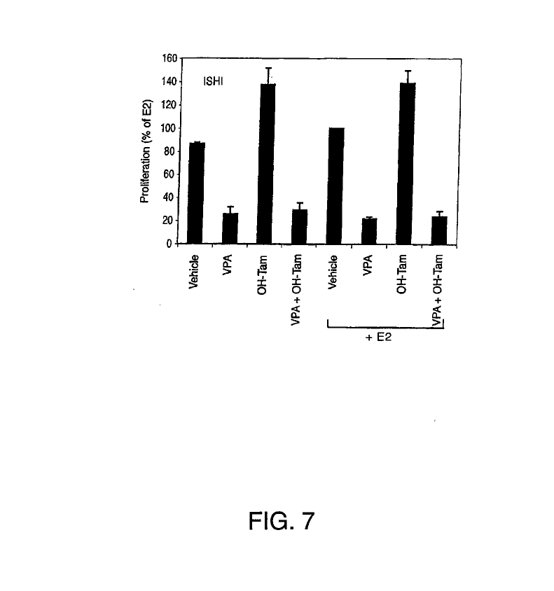

Overexpression of HER2/neu in MCF-7 cells confers tumors grown in nude mice with tamoxifen-resistant growth and also a decreased sensitivity to tamoxifen in vitro (Benz, C. C., et al., Breast Cancer Res Treat., 1993. 24(2): p. 85-95; Kurokawa, H., et al., Cancer Res., 2000. 60(20): p. 5887-94). In the absence of E2, we observed an increased basal level of proliferation in MCF-7/HER2 cells compared to parental MCF-7/neo cells and a slight decrease in sensitivity to tamoxifen in the presence of E2 (FIG. 6). Like parental cells, MCF-7 cells overexpressing HER2/neu are inhibited by VPA and when VPA and tamoxifen treatments are combined, they exhibit a greater decrease in proliferation than when either ligand is used alone, particularly in the presence of E2. These results indicate that VPA enhances the anti-proliferative effects of antiestrogens in both tamoxifen-sensitive cells and in tamoxifen-resistant breast cancer cells.

### Example 7

### VPA Reverses the Agonist Activity of Tamoxifen in Endometrial Cells

Since tamoxifen stimulates the proliferation of uterine endometrial cells, it was unclear whether co-treating with VPA would enhance or antagonize tamoxifen-induced proliferation in endometrial cells. Ishikawa adenocarcinoma cells were treated for 7 days and an increase was observed in cell numbers with both E2 and tamoxifen treatment compared to vehicle alone. VPA dramatically inhibited proliferation and equally antagonized the proliferative effect of E2 and tamoxifen, Thus, while VPA cooperated with the anti-proliferative effect of tamoxifen in breast cells, VPA reversed the estrogen-like agonist activity of tamoxifen in endometrial cells.

### Example 8

### Effective Combinations in Complex Combinations

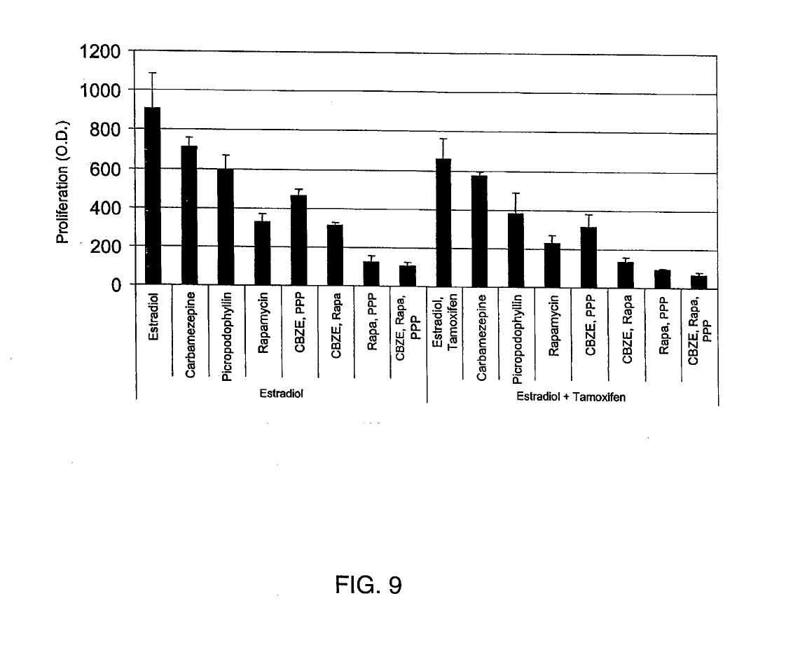

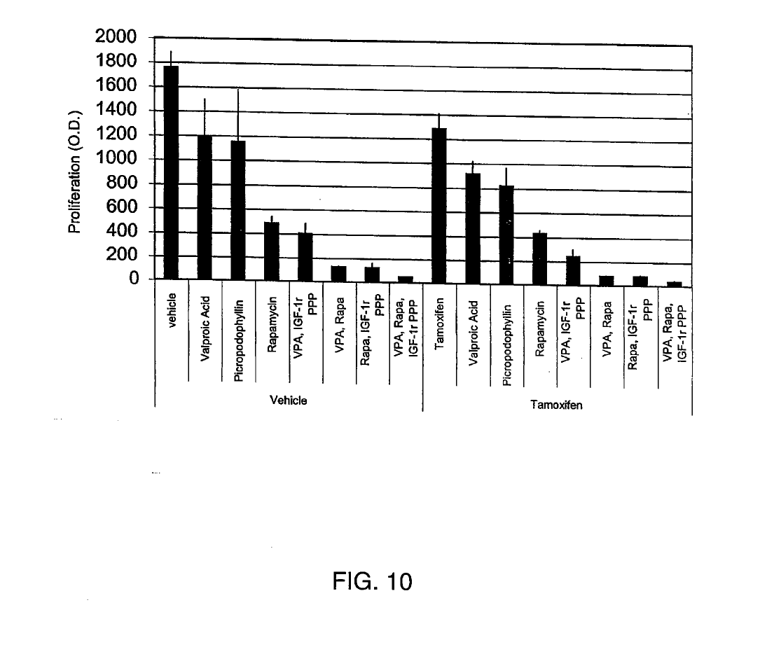

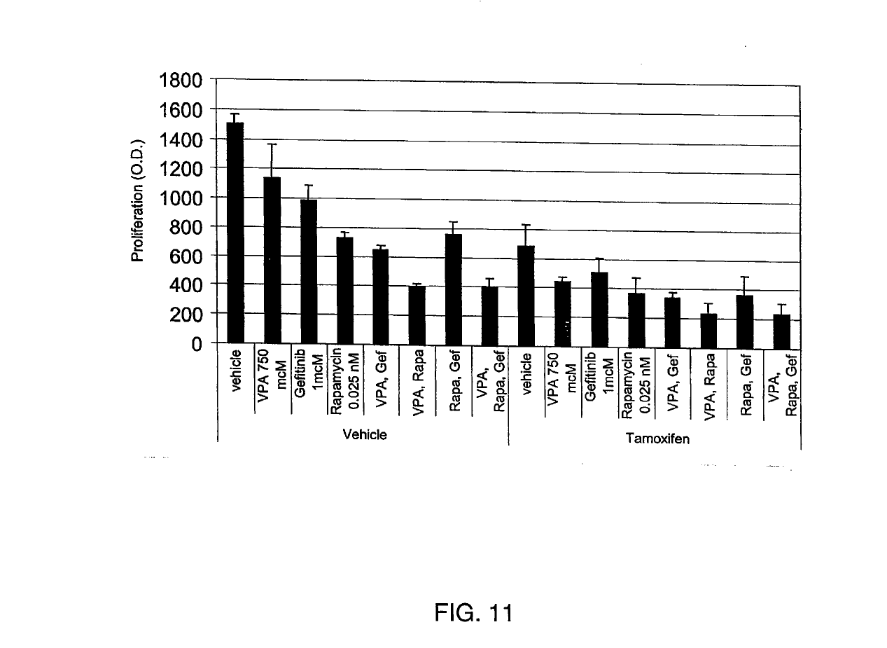

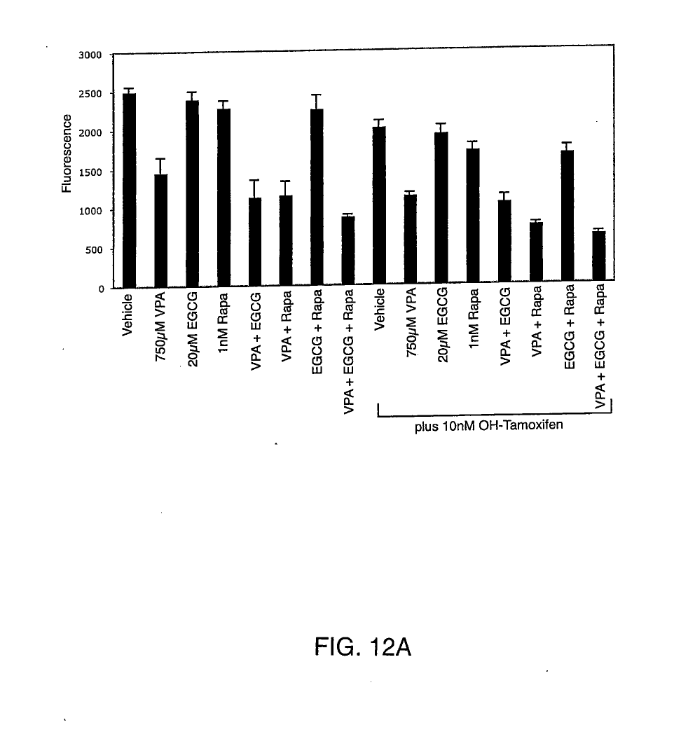

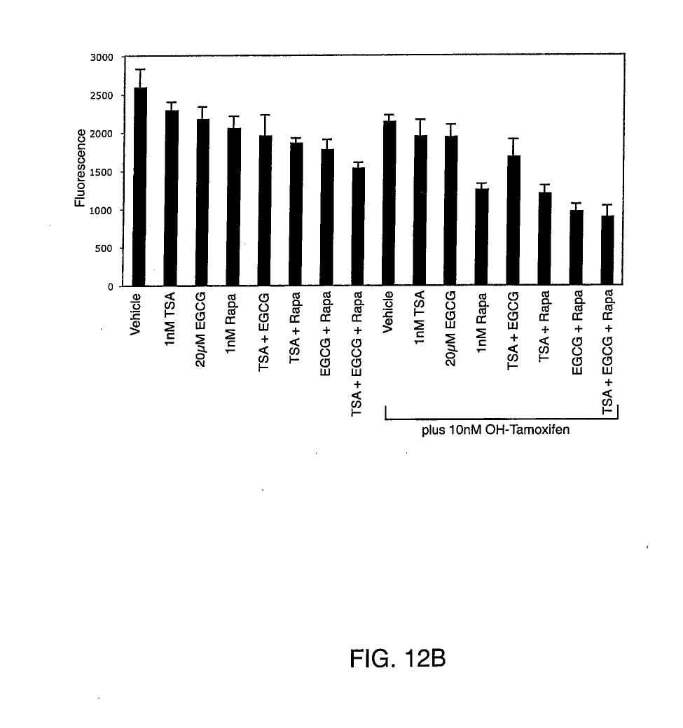

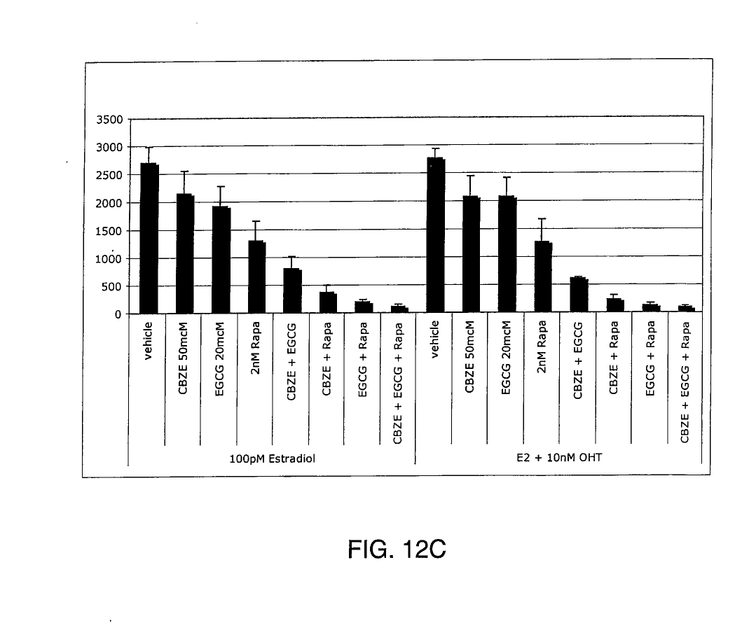

In several experiments, the ability of more complex combinations (cocktails) of HDAC inhibitors along with one or more other agents to prevent proliferation of breast cancer cells either alone or with antiestrogen agents was assessed in MCF-7 breast cancer cells. The action of complex combinations cannot be anticipated from the action of the individual components. Some combinations cooperate efficiently and others do not. As illustrated in the experiments shown in FIGS. 9, 10, and 12, four drugs, an HDAC inhibitor, an IGF-1R inhibitor, an mTOR inhibitor, and an antiestrogen agent were tested in every possible combination. The figures differ by the choice of drug to represent the HDAC inhibitor or the IGF-1R inhibitor; carbamazepine is the HDAC inhibitor and picropodophyllin is the IGF-1R inhibitor for FIG. 9, VPA is the HDAC inhibitor and picropodophyllin is the IGF-1R inhibitor for FIG. 10, and carbamazepine is the HDAC inhibitor and EGCG is the IGF-1R inhibitor for FIG. 12. The Experiments in FIG. 11 use VPA as the HDAC inhibitor, and use combinations including the EGFR inhibitor gefitinib. In each test of four drugs, there are 6 possible dual, 4 triple, and one quadruple combination. In the experiments that generated the data for FIGS. 9, 10, and 12 all of these combinations show additive effects, thus each of the pairs gives more inhibition of breast cancer cell proliferation than either of the single components, each of the triples more than the doubles that can be formed from its components, and the quadruple more inhibition than any of the triples. Effective combination in inhibiting breast cancer cell proliferation was observed for the combinations of an HDAC inhibitor (VPA or carbamazepine) and an antiestrogen (tamoxifen), an HDAC inhibitor (VPA or carbamazepine) and an IGF-1R inhibitor (picropodophyllin or EGCG), and an HDAC inhibitor (VPA or carbamazepine) and an mTOR inhibitor (rapamycin). Effective combination was also observed for each of the four triple combinations and the quadruple combination in the experiments shown in FIGS. 9, 10, and 12. In FIG. 11, four drugs, an HDAC inhibitor, an EGFR inhibitor, an mTOR inhibitor, and an antiestrogen agent, were tested in every possible combination. FIG. 11 demonstrates the effective combination in inhibiting breast cancer cell proliferation observed for the combinations of VPA and gefitinib, or rapamycin, either without or with tamoxifen. In contrast, gefitinib and rapamycin do not combine efficiently—the combination of the two agents yields no more inhibition than does rapamycin alone either in the absence or presence of tamoxifen. Thus, these outcomes could not have been anticipated simply from the fact that each of these agents combined effectively with tamoxifen.

### Example 9

Example 9 illustrates that in MCF-7 cells, the HDAC inhibitor carbamazepine (50 μM) combines effectively with the mTOR inhibitor, rapamycin (2 nM), with picropodophyllin (100 nM), an IGF-1R inhibitor, and with tamoxifen (10 nM), to slow the growth of breast cancer cells (FIG. 9).

### Example 10

Example 10 illustrates that in MCF-7 cells, the HDAC inhibitor VPA (750 μM) combines effectively with the mTOR inhibitor, rapamycin (0.05 nM), with picropodophyllin (100 nM), an IGF-1R inhibitor, and with tamoxifen (100 nM), to slow the growth of breast cancer cells (FIG. 10). The components other than tamoxifen combines effectively with each other and with tamoxifen.

### Example 11

Example 11 illustrates that in MCF-7 cells, the HDAC inhibitor VPA (750 μM) combines effectively with the mTOR inhibitor, rapamycin (0.025 nM), and with gefitinib (1 μM), an EGFR inhibitor, with and without tamoxifen (100 nM), to slow the growth of breast cancer cells (FIG. 11).

### Example 12

Example 12 illustrates that in MCF-7 cells, combinations of HDAC inhibitors with (−)-epigallocatechin-3-gallate (EGCG), rapamycin, and tamoxifen are more efficacious than treatment with any single agent alone to slow the growth of breast cancer cells. See FIG. 12A: The HDAC inhibitor is valproic acid (750 μM), EGCG was used at 20 μM, rapamycin was used at 1 nM, and OH-tamoxifen was used at 10 nM. See FIG. 12B: The HDAC inhibitor is trichostatin A (1 nM), EGCG was used at 20 μM, rapamycin was used at 1 nM, and OH-tamoxifen was used at 10 nM. See FIG. 12C: The HDAC inhibitor, carbamazepine (50 μM), EGCG was used at 20 μM, rapamycin was used at 2 nM, and OH-tamoxifen was used at 10 nM.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating estrogen receptor positive breast cancer, the method comprising the step of administering to a subject a therapeutically effective amount of an HDAC inhibitor in combination with a course of hormonal therapy, wherein the HDAC inhibitor is not valproic acid.

2. The method of claim 1, wherein the HDAC inhibitor is not carbamazepine.

3. The method of claim 1, wherein the hormonal therapy is selected from the group consisting of anti-estrogen therapy and estrogen ablation therapy.

4. The method of claim 3, wherein the estrogen ablation therapy is an aromatase inhibitor.

5. The method of claim 4, wherein the aromatase inhibitor is selected from the group consisting of exemestane, letrozole, and anastrozole.

6. The method of claim 1, wherein the hormonal therapy is an aromatase inhibitor which is administered to the subject in an amount insufficient to fully prevent production of estrogen.

7. The method of claim 3, wherein the anti-estrogen therapy is selected from the group consisting of tamoxifen, raloxifene, fulvestrant, and torimefene.

8. The method of claim 7, wherein the anti-estrogen therapy is tamoxifen.

9. The method of claim 8, wherein the dose of tamoxifen is from about 10 mg/day to about 40 mg/day.

10. The method of claim 7, wherein less than estrogen receptor-saturating amounts of fulvestrant are administered to the subject.

11. The method of claim 1, wherein the HDAC inhibitor is selected from the group consisting of carbamazepine, TSA and SAHA.

12. The method of claim 1, wherein the HDAC inhibitor is administered every day and the hormonal therapy is administered every day.

13. The method of claim 1, wherein the HDAC inhibitor is administered every other day and the hormonal therapy is administered every day.

14. The method of claim 1, wherein the HDAC inhibitor and the hormonal therapy are administered concurrently.

15. The method of claim 1, wherein the HDAC inhibitor and the hormonal therapy are administered separately.

16. The method of claim 3, wherein the HDAC inhibitor is carbamazepine and the estrogen ablation therapy is letrozole.

17. The method of claim 3, wherein the HDAC inhibitor is carbamazepine and the anti-estrogen therapy is selected from the group consisting of tamoxifen, raloxifene, fulvestrant, and torimefene.

18. The method of claim 3, wherein the HDAC inhibitor is selected from the group consisting of carbamazepine, SAHA and TSA and the anti-estrogen therapy is tamoxifen.

19. The method of claim 16, wherein the dose of carbamazepine is from about 200 mg/day to about 600 mg/day.

20. The method of claim 16, wherein the dose of letrozole is from about 1 mg/day to about 5 mg/day.

21. The method of claim 1, wherein the breast cancer is tamoxifen-resistant.

22. The method of claim 1, wherein the breast cancer overexpresses Her2/Neu.

23. The method of claim 1, wherein the subject has failed previous therapy.

24. The method of claim 1, wherein the cancer is recurring.

25. The method of claim 1, wherein the subject is post-menopausal.

26. The method of claim 1, wherein the subject is at risk for breast cancer.

27. The method of claim 1, wherein the subject has Stage 1, Stage 2, Stage 3, or Stage 4 cancer.

28. A pharmaceutical composition comprising a therapeutically effective amount of an HDAC inhibitor in combination with a therapeutically effective amount of a hormonal therapy compound, wherein the HDAC inhibitor is not valproic acid.

29. The composition of claim 28, wherein the HDAC inhibitor is not carbamazepine.

30. The composition of claim 28, wherein the hormonal therapy compound is selected from the group consisting of an anti-estrogen therapy compound and an estrogen ablation therapy compound.

31. The composition of claim 30, wherein the estrogen ablation therapy compound is an aromatase inhibitor.

32. The composition of claim 31, wherein the aromatase inhibitor is selected from the group consisting of exemestane, letrozole, and anastrozole.

33. The composition of claim 31, wherein the aromatase inhibitor is present in an amount insufficient to fully prevent production of estrogen.

34. The composition of claim 30, wherein the anti-estrogen therapy compound is selected from the group consisting of tamoxifen, raloxifene, fulvestrant, and torimefene.

35. The composition of claim 30, wherein the anti-estrogen therapy is tamoxifen.

36. The composition of claim 35, wherein the dose of tamoxifen is from about 10 mg/day to about 40 mg/day.

37. The composition of claim 34, comprising less than estrogen receptor-saturating amounts of fulvestrant.

38. The composition of claim 28, wherein the HDAC inhibitor is selected from the group consisting of carbamazepine, TSA, and SAHA.

39. The composition of claim 28, wherein the HDAC inhibitor is carbamazepine and the estrogen ablation therapy compound is letrozole.

40. The composition of claim 39, wherein the dose of carbamazepine is from about 200 mg/day to about 600 mg/day.

41. The composition of claim 39, wherein the dose of letrozole is from about 1 mg/day to about 5 mg/day.

42. The composition of claim 30, wherein the HDAC inhibitor is carbamazepine and the anti-estrogen therapy compound is selected from the group consisting of tamoxifen, raloxifene, fulvestrant, and torimefene.

43. The composition of claim 30, wherein the HDAC inhibitor is selected from the group consisting of carbamazepine, SAHA and TSA and the anti-estrogen therapy compound is tamoxifen.

44. The composition of claim 42, wherein the dose of tamoxifen is from about 10 mg/day to about 40 mg/day.

45. The composition of claim 28, wherein the composition is formulated for parenteral or oral administration.

46. A method of preventing estrogen receptor positive breast cancer, the method comprising the step of administering to a subject a therapeutically effective amount of an HDAC inhibitor in combination with a course of anti-estrogen therapy or estrogen ablation therapy.

47. The method of claim 46, wherein there is no attendant increase in the risk of uterine cancer when the anti-estrogen therapy is tamoxifen or raloxifen as compared to treatment with tamoxifen and raloxifene alone.

48. The method of claim 46, wherein the subject is in remission from breast cancer.

49. The method of claim 46, wherein the subject has previously undergone treatment.

50. The method of claim 46, wherein the breast cancer is prevented from progressing from DCIS.

51. The method of claim 46, wherein the breast cancer is prevented from progressing from atypical hyperplasia.

52. The method of claim 46, wherein the anti-estrogen therapy is selected from the group consisting of tamoxifen and raloxifene, and the HDAC inhibitor is selected from the group consisting of valproic acid, carbamazepine, TSA, and SAHA.

53. The method of claim 46, wherein the estrogen ablation therapy is selected from the group consisting of exemestane, letrozole, and anastrozole.

54. The method of claim 52, wherein the ratio of valproic acid to tamoxifen is from about 1 part valproic acid to from about 22.5 to about 180 parts tamoxifen.

55. The method of claim 46, wherein the subject has a genetic predisposition to breast cancer.

56. The method of claim 46, wherein the subject has undergone surgery to remove a primary tumor.

57. A method of treating breast cancer, the method comprising the step of administering to a subject a therapeutically effective amount of an HDAC inhibitor in combination with a therapeutically effective amount of at least one compound selected from the group consisting of an IGF-1R inhibitor, an mTOR inhibitor, and an EGFR inhibitor, wherein the HDAC inhibitor is not valproic acid when the compound is an EGFR inhibitor.

58. The method of claim 57, wherein the IGF-1R inhibitor is selected from the group consisting of picropodophyllin and EGCG.

59. The method of claim 57, wherein the mTOR inhibitor is selected from the group consisting of rapamycin and rapamycin derivatives.

60. The method of claim 57, wherein the EGFR inhibitor is gefitinib.

61. The method of claim 57, comprising the step of administering to a subject a therapeutically effective amount of an HDAC inhibitor in combination with a therapeutically effective amount of an IGF-1R inhibitor and an mTOR inhibitor.

62. The method of claim 61, wherein the breast cancer is estrogen receptor positive and the compounds are administered further in combination with a course of hormonal therapy.

63. A pharmaceutical composition comprising a therapeutically effective amount of an HDAC inhibitor in combination with a therapeutically effective amount of at least one compound selected from the group consisting of an IGF-1R inhibitor, an mTOR inhibitor, and an EGFR inhibitor, wherein the HDAC inhibitor is not valproic acid when the compound is an EGFR inhibitor.

64. The composition of claim 63, wherein the HDAC inhibitor is valproic acid.

65. The composition of claim 63, wherein the HDAC inhibitor is carbamazepine.

66. The composition of claim 63, wherein the HDAC inhibitor is valproic acid or carbamazepine and the IGF-1R inhibitor is picropodophyllin.

67. The composition of claim 63, wherein the HDAC inhibitor is valproic acid or carbamazepine and the EGFR inhibitor is gefitinib.

68. The composition of claim 63, wherein the HDAC inhibitor is valproic acid or carbamazepine and the mTOR inhibitor is rapamycin.

69. The composition of claim 63, wherein the HDAC inhibitor is valproic acid or carbamazepine and the mTOR inhibitor is rapamycin.

70. The composition of claim 63, comprising a therapeutically effective amount of an HDAC inhibitor in combination with a therapeutically effective amount of an IGF-1R inhibitor and an mTOR inhibitor.

71. The composition of claim 63 or 70, wherein the compositions further comprise compounds for hormonal therapy.

72. The composition of claim 71, wherein the HDAC inhibitor is valproic acid or carbamazepine, the IGF-1R inhibitor is EGCG, and the hormonal therapy is tamoxifen.

73. The composition of claim 72, wherein the dose of EGCG is from about 300 mg/day to about 800 mg/day.

74. A method of treating estrogen receptor positive breast cancer, the method comprising the step of administering to a subject a therapeutically effective amount of an HDAC inhibitor in combination with a course of hormonal therapy and one or more additional active ingredients effective to treat estrogen receptor positive breast cancer in the combination.

75. The method of claim 74, wherein the HDAC inhibitor is carbamazepine.

76. The method of claim 75, wherein the dose of carbamazepine is from about 200 mg/day to about 600 mg/day.

77. The method of claim 74, wherein the HDAC inhibitor is valproic acid.

78. The method of claim 77, wherein the dose of valproic acid is from about 300 to about 1000 micromolar in patient serum.

79. The method of claim 77, wherein the dose of valproic acid is from about 500 to about 1000 micromolar in patient serum.

80. The method of claim 74, wherein the one or more additional active ingredients are selected from the group consisting of an IGF-1R inhibitor, an mTOR inhibitor, and an EGFR inhibitor.

81. The method of claim 74, wherein the hormonal therapy is selected from the group consisting of tamoxifen, letrozole, and torimefene.

82. A pharmaceutical composition comprising a therapeutically effective amount of an HDAC inhibitor in combination with a therapeutically effective amount of a hormonal therapy compound and one or more additional active ingredients.

83. The composition of claim 82, wherein the HDAC inhibitor is valproic acid.

84. The composition of claim 82, wherein the HDAC inhibitor is carbamazepine.

85. The composition of claim 82, wherein the one or more additional active ingredients are selected from the group consisting of an IGF-1R inhibitor, an mTOR inhibitor, and an EGFR inhibitor.

86. The composition of claim 83, where in the hormonal therapy is tamoxifen.

87. The composition of claim 86, wherein the additional ingredient is EGCG or rapamycin.
